(19) United States
(12) Patent Application Publication
(10) Pub. No.: US 2020/0377505 A1
(43) Pub. Date: Dec. 3, 2020

(54) BENZIMIDAZOLES AND AZA-BENZIMIDAZOLES, AND METHODS OF USE THEREOF

(71) Applicant: Goldfinch Bio., Inc., Cambridge, MA (US)

(72) Inventors: Mark W. Ledeboer, Acton, MA (US); Jean-Christophe P. Harmange, Andover, MA (US); Maolin Yu, West Roxbury, MA (US); Thomas T. Tibbitts, Westford, MA (US); Matthew H. Daniels, Somerville, MA (US)

(21) Appl. No.: 16/636,606

(22) PCT Filed: Aug. 3, 2018
(86) PCT No.: PCT/US18/45090

§ 371 Date: Feb. 4, 2020

Related U.S. Application Data
(60) Provisional application No. 62/541,457, filed on Aug. 4, 2017.

(51) Int. Cl. C07D 487/04; C07D 235/04; C07D 235/32; C07D 409/12; C07D 403/12; C07D 413/12; C07D 409/14; C07D 401/12; C07D 401/06; C07D 405/04; C07D 417/14; C07D 413/06

(52) U.S. Cl. CPC C07D 487/04; C07D 235/04; C07D 235/32; C07D 409/12; C07D 403/12; C07D 413/06; C07D 409/14; C07D 401/12; C07D 401/06; C07D 405/04; C07D 417/14; C07D 413/12

(57) ABSTRACT

Disclosed are compounds according to Formula (I) or (II), and pharmaceutical compositions comprising them. Also disclosed are therapeutic methods, e.g., of treating kidney diseases, using the compounds of Formula (I) or (II).

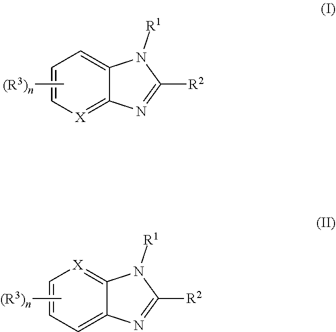

### RELATED APPLICATION

This application claims benefit of priority to U.S. Provisional Patent Application Ser. No. 62/541,457, filed Aug. 4, 2017.

### BACKGROUND

Proteinuria is a condition in which an excessive amount of protein in the blood leaks into the urine. Proteinuria can progress from a loss of 30 mg of protein in the urine over a 24-hour period (called microalbuminuria) to >300 mg/day (called macroalbuminuria), before reaching levels of 3.5 grams of protein or more over a 24-hour period, or 25 times the normal amount. Proteinuria occurs when there is a malfunction in the kidney's glomeruli, causing fluid to accumulate in the body (edema). Prolonged protein leakage has been shown to result in kidney failure. Nephrotic Syndrome (NS) disease accounts for approximately 12% of prevalent end stage renal disease cases at an annual cost in the United States of more than $3 billion. Approximately 5 out of every 100,000 children are diagnosed with NS every year and 15 out of every 100,000 children are living with it today. For patients who respond positively to treatment, the relapse frequency is extremely high. 90% of children with Nephrotic Syndrome will respond to treatment, however, an estimated 75% will relapse. Therefore, more effective methods of treating, or reducing risk of developing, kidney disease, e.g., proteinuria, are desirable.

Mammalian TRP channel proteins form six-transmembrane cation-permeable channels that may be grouped into six subfamilies on the basis of amino acid sequence homology (TRPC, TRPV, TRPM, TRPA, TRPP, and TRPML). Recent studies of TRP channels indicate that they are involved in numerous fundamental cell functions and are considered to play an important role in the pathophysiology of many diseases. Many TRPs are expressed in kidney along different parts of the nephron and growing evidence suggest that these channels are involved in hereditary, as well as acquired kidney disorders. TRPC6, TRPM6, and TRPP2 have been implicated in hereditary focal segmental glomerulosclerosis (FSS), hypomagnesemia with secondary hypocalcemia (HSH), and polycystic kidney disease (PKD), respectively.

TRPC5 has also been reported to contribute to the mechanisms underlying regulation of innate fear responses. (J Neurosci. 2014 Mar. 5; 34(10): 3653-3667).

Hence, there is a need for additional inhibitors of TRPC5.

### SUMMARY

This invention is based, at least in part, on the discovery that Transient Receptor Potential Cation Channel, subfamily C, member 5 (TRPC5) activity abolishes actin stress fibers and diminishes focal adhesion formation, rendering a motile, migratory podocyte phenotype.

In one aspect, the invention relates to methods of treating, or reducing risk of developing, kidney disease (e.g., proteinuria, microalbuminuria, macroalbuminuria), anxiety, depression, or cancer, in a subject by administering a therapeutically effective amount of a TRPC5 inhibitor to the subject.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

In some embodiments, the methods include administering a small molecule that inhibits TRPC5.

In some embodiments, the invention relates to a compound of Formula (I) or (II) or a pharmaceutically acceptable salt thereof;

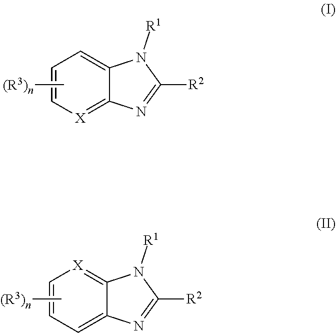

wherein

X is CH, C(R³), or N;

R¹ is selected from the group consisting of H; alkyl; cycloalkyl; heterocycloalkyl; alkenyl; aryl; heteroaryl; alkylene-aryl; alkylene-heteroaryl; —CH₂(O)N(R)-heteroaryl; —CH₂(O)N(R)-alkyl; alkylene-N(alkyl)₂; heterocycloalkyl; alkylene-O-alkyl; alkylene-O-aryl; alkylene-N(R)—C(O)-aryl; alkylene-N(R)—C(O)-alkyl; alkylene-C(O)—N(R)-alkyl; alkylene-C(O)—N(R)-aryl; alkylene-C(O)-cycloalkyl; and alkylene-C(O)—N(R)-heteroaryl;

R² is selected from the group consisting of H; NH₂; alkyl; cycloalkyl; aryl; heteroaryl; alkylene-aryl; alkylene-N(alkyl)₂; alkylene-heterocycloalkyl; alkylene-cycloalkyl; —N(R)-alkyl; —N(R)-aryl; —N(R)-alkylene-aryl; —N(R)-cycloalkyl; —N(R)-heterocycloalkyl; —O-aryl; alkylene-O-aryl; heterocycloalkyl; —N═C(R)-aryl; —N(R)-alkylene-heteroaryl; —N(R)-alkylene-OH; —S— alkylene-C(O)N(R)-aryl; —S-alkylene-C(O)N(R)-heteroaryl; alkylene-C(O)-heterocycloalkyl; alkylene-N(R)-alkyl; alkylene-N(R)-aryl; and —S-alkyl;

R³ is independently selected from alkyl, halogen, CN, OMe, OH, NO₂, NH₂, N(Me)₂, CF₃, OCF₃, CHF₂, OCHF₂, and —O-alkylene-OH;

R is H, or Me; and

n is 0, 1, 2, 3, or 4;

provided the compound is not

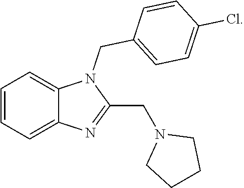 [SMILES: Clc1ccc(Cn2c(CN3CCCC3)nc3ccccc32)cc1]

In one aspect, the invention features a composition, comprising a compound of any one of Formula (I) or (II) or a pharmaceutically acceptable salt thereof; and a pharmaceutically acceptable excipient.

In one aspect, the invention features methods of treating, or the reducing risk of developing, a kidney disease, anxiety, or depression, or cancer, comprising administering to a subject in need thereof a therapeutically effective amount of a compound of Formula (I) or (II). In certain embodiments, a kidney disease is treated or the risk of developing a kidney disease is reduced. In certain embodiments, a kidney disease is treated. In certain embodiments, the kidney disease is selected from the group consisting of Focal Segmental Glomerulosclerosis (FSGS), Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive GN, anti-GBM disease, C3 glomerulonephritis, hypertensive nephrosclerosis, and IgA nephropathy. In certain embodiments, the kidney disease is proteinuria. In certain embodiments, the kidney disease is microalbuminuria or macroalbuminuria.

In certain embodiments, the subject is a mammal. In certain embodiments, the mammal is a human.

In some embodiments, the invention comprises administering the compound of Formula (I) or (II) to a mammal and evaluating an effect of the compound on calcium transport, wherein a compound that reduces or inhibits calcium transport is a therapeutic agent for treating or reducing risk of developing a kidney disease, anxiety, depression, or cancer.

The invention provides several advantages. The prophylactic and therapeutic methods described herein are effective in treating kidney disease, e.g., proteinuria, and have minimal, if any, side effects. Further, methods described herein are effective to identify compounds that treat or reduce risk of developing a kidney disease, anxiety, depression, or cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 tabulates characterization data for representative compounds of the invention.

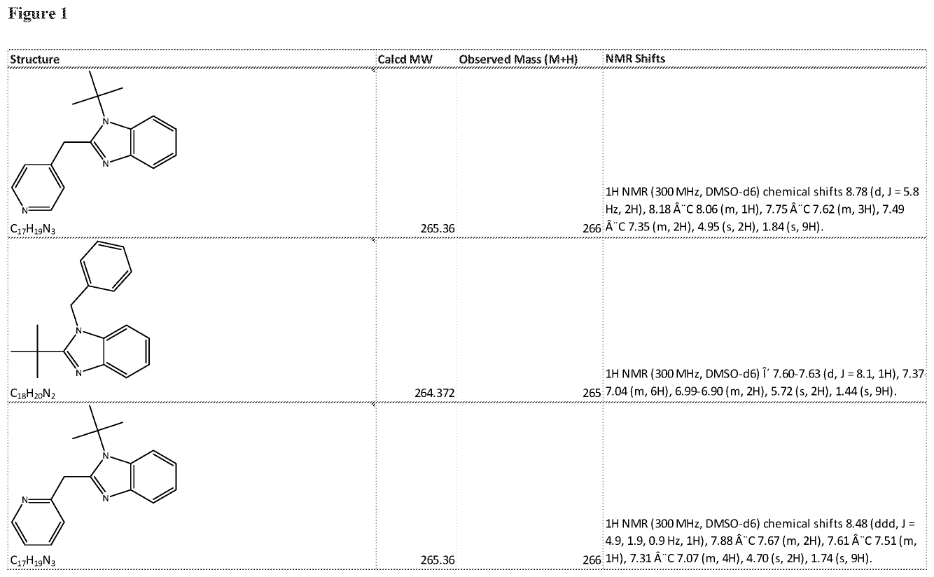

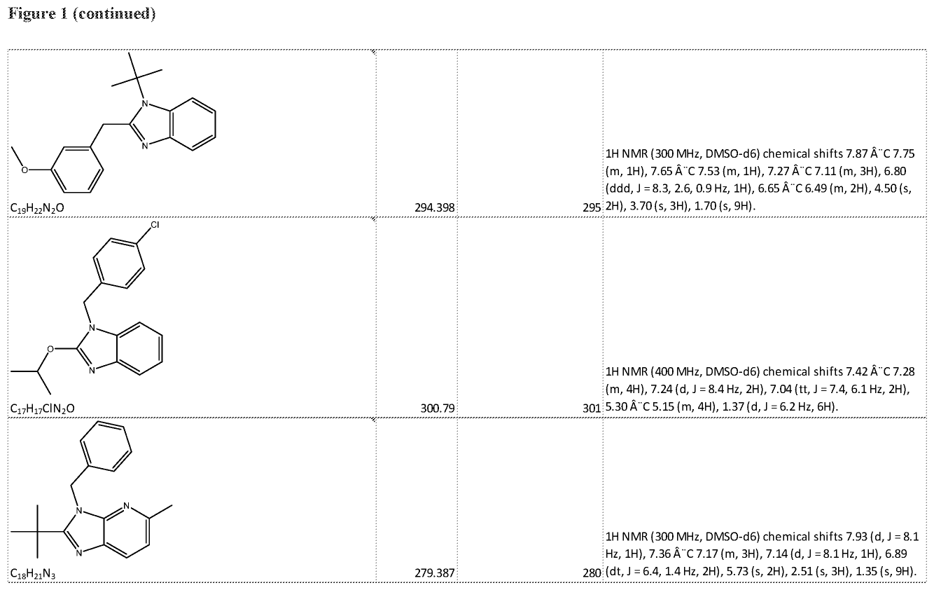

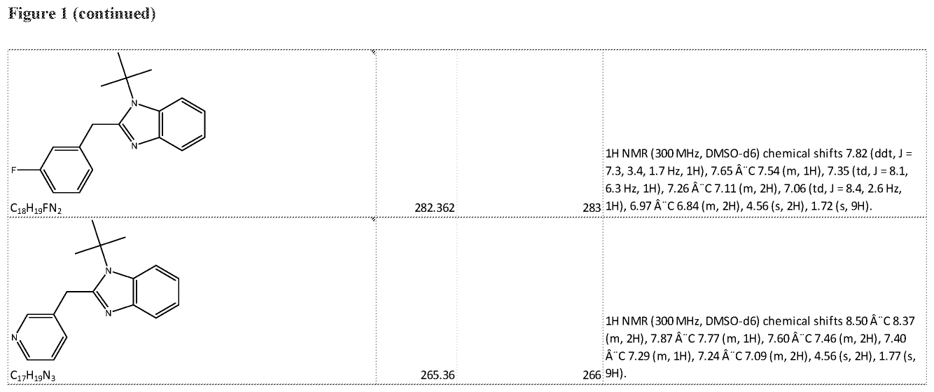

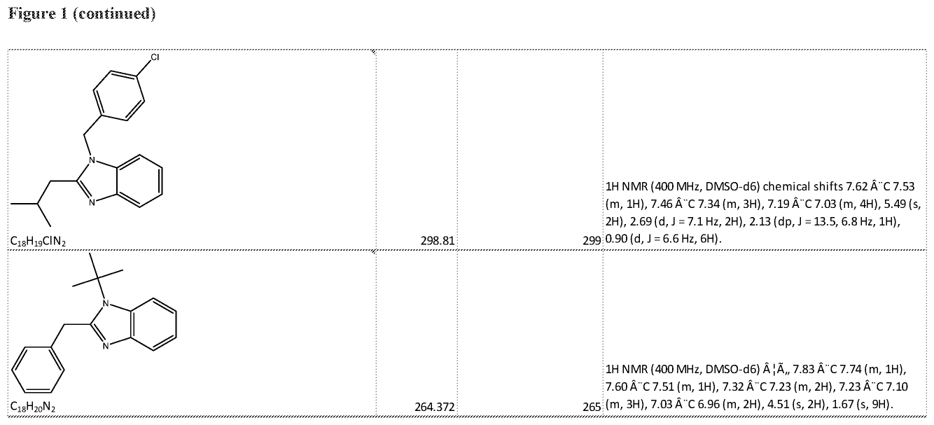

### DETAILED DESCRIPTION

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkyC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless otherwise specified, “alkylene” by itself or as part of another substituent refers to a saturated straight-chain or branched divalent group having the stated number of carbon atoms and derived from the removal of two hydrogen atoms from the corresponding alkane. Examples of straight chained and branched alkylene groups include —CH₂— (methylene), —CH₂—CH₂— (ethylene), —CH₂—CH₂—CH₂— (propylene), —C(CH₃)₂—, —CH₂—CH(CH₃)—, —CH₂—CH₂—CH₂—CH₂— —CH₂—CH₂—CH₂—CH₂—CH₂— (pentylene), —CH₂—CH(CH₃)—CH₂—, and —CH₂—C(CH₃)₂—CH₂—.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkyS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

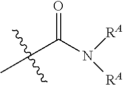

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

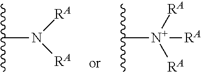

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, aryls, heteroaryls, and/or heterocyclyls. Aryl groups include benzene, naphthalene, phenanthrene, phenol, aniline, and the like.

The term “carbamate” is art-recognized and refers to a group

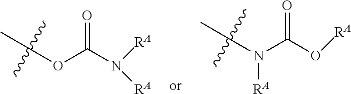

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl” or “heterocycloalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

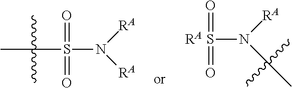

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

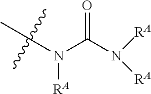

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the parent compound is presented as an ester.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required effectively to treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

Compounds of the Invention

One aspect of the invention provides small molecule inhibitors of TRPC5.

In some embodiments, the compound of the invention is a compound of Formula (I) or (II) or a pharmaceutically acceptable salt thereof;

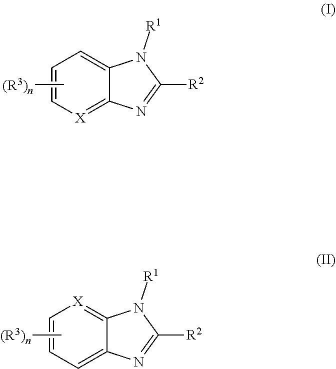

wherein

X is CH, C(R³), or N;

R¹ is selected from the group consisting of H; alkyl; cycloalkyl; heterocycloalkyl; alkenyl; aryl; heteroaryl; alkylene-aryl; alkylene-heteroaryl; —CH₂(O)N(R)-heteroaryl; —CH₂(O)N(R)-alkyl; alkylene-N(alkyl)₂; heterocycloalkyl; alkylene-O-alkyl; alkylene-O-aryl; alkylene-N(R)—C(O)-aryl; alkylene-N(R)—C(O)-alkyl; alkylene-C(O)—N(R)-alkyl; alkylene-C(O)—N(R)-aryl; alkylene-C(O)-cycloalkyl; and alkylene-C(O)—N(R)-heteroaryl;

R² is selected from the group consisting of H; NH₂, alkyl; cycloalkyl; aryl; alkylene-aryl, alkylene-N(alkyl)₂; alkylene-heterocycloalkyl; alkylene-cycloalkyl; —N(R)-alkyl; —N(R)-aryl; —N(R)-alkylene-aryl; —N(R)-cycloalkyl; —N(R)-heterocycloalkyl; —O-aryl; alkylene-O-aryl; heterocycloalkyl; —N═C(R)-aryl; —N(R)-alkylene-heteroaryl; —N(R)-alkylene-OH; —S-alkylene-C(O)N(R)-aryl; —S-alkylene-C(O)N(R)-heteroaryl; alkylene-C(O)-heterocycloalkyl; alkylene-N(R)-alkyl; alkylene-N(R)-aryl; and —S-alkyl;

R³ is independently selected from alkyl, halogen, CN, OMe, OH, NO₂, NH₂, N(Me)₂, CF₃, OCF₃, CHF₂, OCHF₂, and —O-alkylene-OH;

R is H, or Me; and

n is 0, 1, 2, 3, or 4;

provided the compound is not

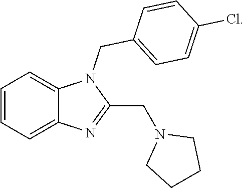 [SMILES: Clc1ccc(Cn2c(CN3CCCC3)nc3ccccc32)cc1]

In some embodiments, X is CH. In some embodiments, X is N. In some embodiments, X is C(R³).

In some embodiments, R is selected from the group consisting of H, methyl, ethyl, n-propyl, i-propyl, n-butyl, i-butyl, and t-butyl. In some embodiments, R¹ is cycloalkyl or heterocycloalkyl.

In some embodiments, R is aryl or heteroaryl.

In some embodiments, R¹ is alkylene-aryl. In some embodiments, alkylene-aryl is methylene-aryl. In some embodiments, alkylene-phenyl is alkylene-substituted phenyl. In some embodiments, substituted phenyl is substituted with one or more substituents independently selected from is alkyl, halogen, OMe, OH, NO₂, NH₂, N(Me)₂, CF₃, OCF₃, CHF₂, OCHF₂, and —O-alkylene-OH.

In some embodiments, R¹ is alkylene-heteroaryl. In some embodiments, alkylene-aryl is methylene-phenyl. In some embodiments, alkylene-heteroaryl is methylene-pyridine.

In some embodiments, R¹ is selected from —CH₂(O)N(R)-heteroaryl; —CH₂(O)N(R)-alkyl.

In some embodiments, R¹ is —CH₂(O)N(H)-heteroaryl. In some embodiments, R¹ is —CH₂(O)N(H)-oxazole, wherein the oxazole is substituted.

In some embodiments, R¹ is —CH₂(O)N(H)-alkyl. In some embodiments, R¹ is —CH₂(O)N(H)-t-butyl.

In some embodiments, R¹ is alkylene-N(Me)₂ or alkylene-N(Et)₂. In some embodiments, alkylene-N(Me)₂ is ethylene-N(Me)₂. In some embodiments, alkylene-N(Et)₂ is ethylene-N(Et)₂.

In some embodiments, R¹ is heterocycloalkyl. In some embodiments, heterocycloalkyl is methyl-piperidinyl.

In some embodiments, R¹ is alkenyl. In some embodiments, R¹ is substituted aryl. In some embodiments, R¹ is alkylene-O-alkyl. In some embodiments, R¹ is alkylene-O-aryl. In some embodiments, R¹ is alkylene-C(O)-cycloalkyl

In some embodiments, R¹ is alkylene-N(H)—C(O)-aryl. In some embodiments, R¹ is alkylene-N(H)—C(O)-alkyl. In some embodiments, R¹ is alkylene-C(O)—N(H)-alkyl. In some embodiments, R¹ is alkylene-C(O)—N(H)-aryl. In some embodiments, R¹ is alkylene-C(O)—N(H)-heteroaryl.

In some embodiments, aryl or heteroaryl is substituted with one or more substituent. In some embodiments, the substituent is alkyl, halogen, OMe, OH, NO₂, NH₂, N(Me)₂, CF₃, OCF₃, CHF₂, OCHF₂, or —O-alkylene-OH.

In some embodiments, R² is NH₂. In some embodiments, R² is alkylene-aryl. In some embodiments, alkylene-aryl is methylene-phenyl.

In some embodiments, R² is alkylene-N(Me)₂. In some embodiments, alkylene-N(Me)₂ is ethylene-N(Me)₂.

In some embodiments, R² is —N(H)-alkyl. In some embodiments, —N(H)-alkyl is —N(H)— methyl.

In some embodiments, R² is —N(H)-aryl. In some embodiments, —N(H)-aryl is —N(H)— substituted phenyl.

In some embodiments, R² is —N(H)-cycloalkyl. In some embodiments, cycloalkyl is a 3-6 membered ring. In some embodiments, cycloalkyl is substituted cycloalkyl. In some embodiments, cyclocalkyl is cyclohexyl. In some embodiments, cyclohexyl is substituted cyclohexyl.

In some embodiments, R² is alkylene-heterocycloalkyl. In some embodiments, alkylene-heterocycloalkyl is alkylene-substituted heterocycloalkyl. In some embodiments, alkylene heterocycloalkyl is

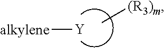

wherein Y is N, O, or S; m is an integer selected from 0, 1, 2, or 3, and

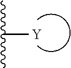

represents a 4-12 membered heterocycle. In some embodiments, the 4-12 membered heterocycle comprising one or more heteroatoms. In some embodiments

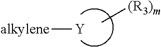

is

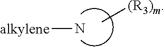

In some embodiments,

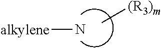

is

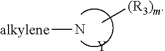

In some embodiments, alkylene heterocycloalkyl is

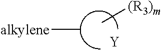

wherein Y is N, O, or S; m is an integer selected from 0, 1, 2, or 3, and

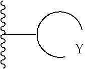

represents a 4-12 membered heterocycle. In some embodiments, the 4-12 membered heterocycle comprising one or more heteroatoms. In some embodiments,

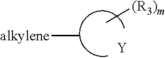

is

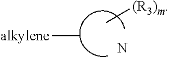

In some embodiments,

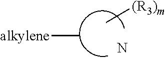

is

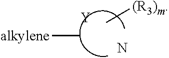

In some embodiments, R² is —N(H)-heterocycloalkyl. In some embodiments, heterocycloalkyl is a 3-6 membered ring. In some embodiments, heterocycloalkyl is substituted heterocycloalkyl. In some embodiments, heterocycloalkyl is tetrahydropyranyl.

In some embodiments, R² is —N(H)-alkylene-aryl. In some embodiments, —N(H)-alkylene-aryl is —N(H)-methylene-aryl. In some embodiments, —N(H)-methylene-aryl is —N(H)-methylene-phenyl. In some embodiments, —N(H)-methylene-phenyl is —N(H)-methylene-substituted phenyl.

In some embodiments substituted phenyl is substituted phenyl with alkyl, halogen, OMe, OH, NO₂, NH₂, N(Me)₂, CF₃, OCF₃, CHF₂, OCHF₂, or —O-alkylene-OH.

In some embodiments, R² is —O-aryl. In some embodiments, —O-phenyl is —O-substituted phenyl. In some embodiments, R² is —O-substituted phenyl. In some embodiments, substituted phenyl is substituted with halogen.

In some embodiments, R² is heterocycloalkyl. In some embodiments, heterocycloalkyl is piperidinyl, pyrrolidinyl, or pyrrolidinonyl. In some embodiments, piperidinyl is substituted piperidinyl. In some embodiments, pyrrolidinyl is substituted pyrrolidinyl. In some embodiments, pyrrolidinonyl is substituted pyrrolidinonyl. In some embodiments, the substitution is alkylene-heteroaryl. In some embodiments, alkylene-heteroaryl is alkylene-substitute heteroaryl. In some embodiments, the substitution is an alkylene-aryl. In some embodiments, the substitution is an alkylene-aryl. In some embodiments, alkylene-aryl is alkylene-substituted aryl.

In some embodiments, R² is H. In some embodiments, alkyl is selected from methyl, ethyl, n-propyl, i-propyl, n-butyl, i-butyl, and t-butyl.

In some embodiments, R² is cycloalkyl. In some embodiments, cycloalkyl is cyclopropyl.

In some wherein R² is aryl. In some embodiments, aryl is phenyl. In some embodiments, phenyl is substituted phenyl.

In some embodiments, R² is —O-aryl. In some embodiments, —O-aryl is —O-substituted aryl. In some embodiments, —O-aryl is —O-phenyl.

In some embodiments, R² is alkylene-O-aryl. In some embodiments, alkylene-O-aryl is -alkylene-O-substituted aryl. In some embodiments, alkylene-O-aryl is methylene-O-phenyl.

In some embodiments, R² is N═C(H)-aryl. In some embodiments, N═C(H)-aryl is N═C(H)-substituted aryl.

In some embodiments, R² is —N(H)-alkylene-heteroaryl. In some embodiments, —N(H)— alkylene-heteroaryl is —N(H)-methylene-heteroaryl. In some embodiments, —N(H)-methylene-heteroaryl is —N(H)-methylene-substituted heteroaryl.

In some embodiments, R² is —N(H)-alkylene-OH. In some embodiments, —N(H)-alkylene-OH is —N(H)-methylene-OH. In some embodiments, —N(H)-alkylene-OH is —N(H)-ethylene-OH. In some embodiments, —N(H)-alkylene-OH is —N(H)-i-propylene-OH.

In some embodiments, R² is —S-alkylene-C(O)N(H)-aryl. In some embodiments, —S— alkylene-C(O)N(H)-aryl is —S-methylene-C(O)N(H)-aryl. In some embodiments, —S-methylene-C(O)N(H)-aryl is —S-methylene-C(O)N(H)-substituted aryl.

In some embodiments, R² is —S-alkylene-C(O)N(H)-heteroaryl. In some embodiments, —S-alkylene-C(O)N(H)-heteroaryl is —S-methylene-C(O)N(H)-heteroaryl. In some embodiments, —S-methylene-C(O)N(H)-heteroaryl is —S-methylene-C(O)N(H)-substituted heteroaryl.

In some embodiments, R² is alkylene-C(O)-heterocycloalkyl. In some embodiments, alkylene-C(O)-heterocycloalkyl is alkylene-C(O)-substituted heterocycloalkyl. In some embodiments substituted heterocycloalkyl is heteroaryl-substituted heterocycloalkyl.

In some embodiments, R² is alkylene-N(H)-alkyl. In some embodiments, alkylene-N(H)-alkyl is alkylene-N(H)— substituted alkyl. In some embodiments, substituted alkyl is cycloalkyl-substituted-alkyl. In some embodiments, alkylene-N(H)-alkyl is substituted-alkylene-N(H)-alkyl. In some embodiments, substituted-alkylene N(H)-alkyl is methyl-substituted-alkylene-N(H)-alkyl.

In some embodiments, R² is alkylene-N(H)-aryl. In some embodiments, alkylene-N(H)-aryl is alkylene-N(H)-substituted aryl. In some embodiments, alkylene-N(H)-aryl is substituted-alkylene-N(H)-aryl. In some embodiments, substituted-alkylene-N(H)-aryl is methyl-substituted-alkylene-N(H)-aryl.

In some embodiments, R² is —S-alkyl.

In some embodiments, R² is methylene-cycloalkyl. In some embodiments, methylene-cycloalkyl is methylene-substituted cycloalkyl.

In some embodiments, R² is methylene-heterocycloalkyl. In some embodiments, methylene-heterocycloalkyl is methylene-substituted heterocycloalkyl.

In some embodiments, R³ is alkyl. In some embodiments, R³ is methyl, ethyl, i-propyl, n-propyl, i-butyl, n-butyl, or t-butyl. In some embodiments, R³ is methyl.

In some embodiments, R³ is halogen. In some embodiments, R³ is F. In some embodiments, R³ is NH₂. In some embodiments, R³ is NO₂. In some embodiments, R³ is N(Me)₂.

In some embodiments, R³ is OMe. In some embodiments, R³ is OH.

In some embodiments, R³ is OCF₃. In some embodiments, R³ is CF₃.

In some embodiments, n is 0.

In some embodiments, n is 1.

In some embodiments, the compound is selected from the group consisting of:

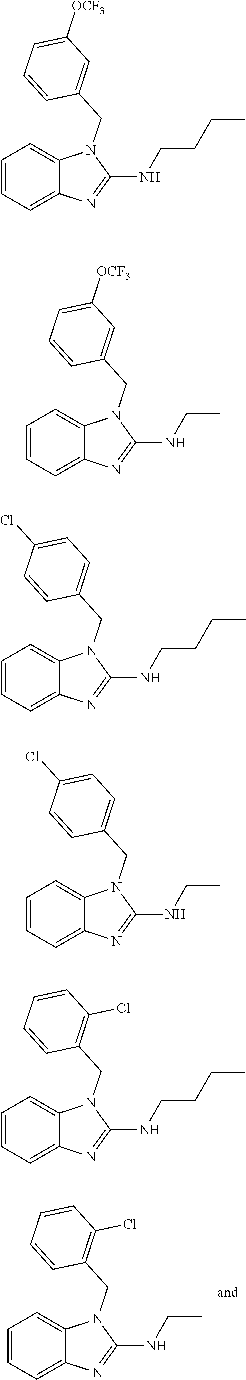

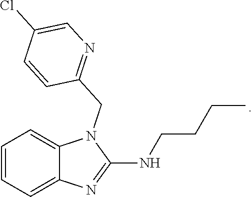 [SMILES: CCCCNc1nc2ccccc2n1Cc1ccc(Cl)cn1]

In some embodiments, the compound is selected from the group consisting of:

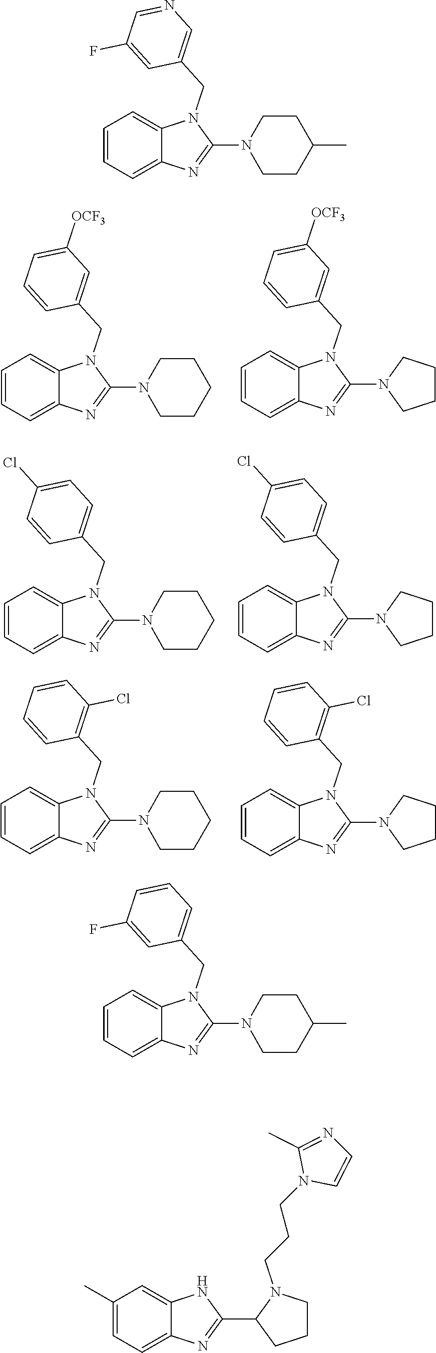

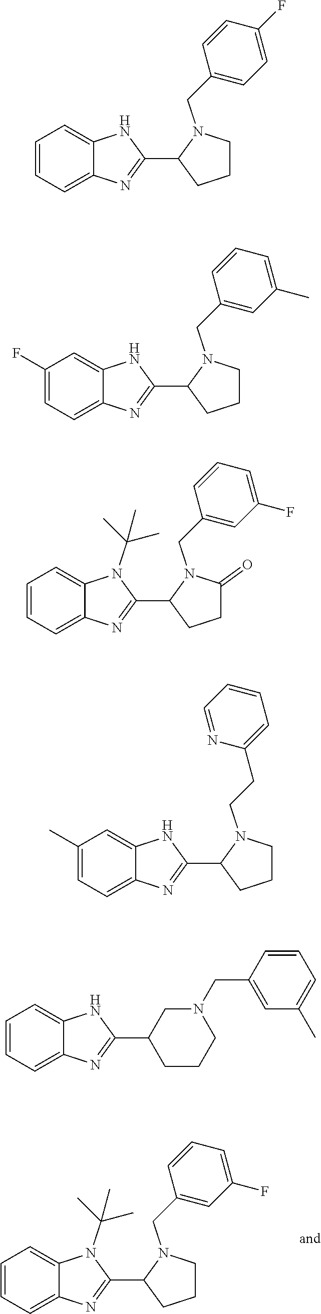

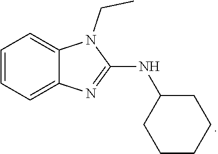 [SMILES: CCn1c(NC2CCCCC2)nc2ccccc21]

In some embodiments, the compound is selected from the group consisting of:

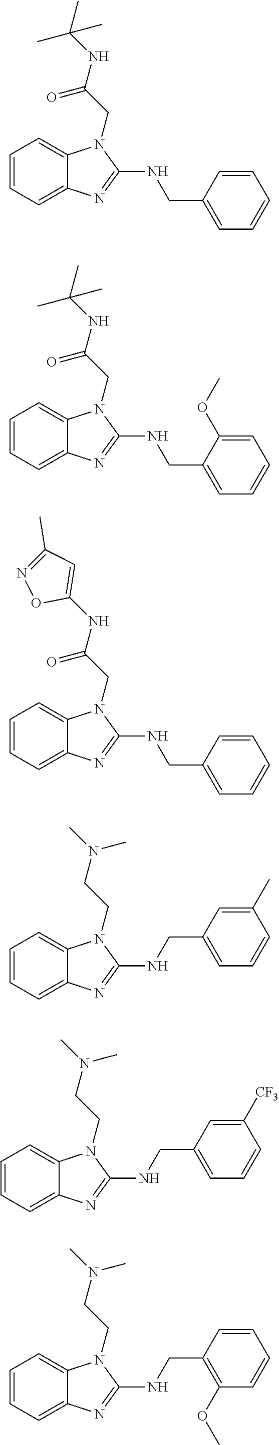

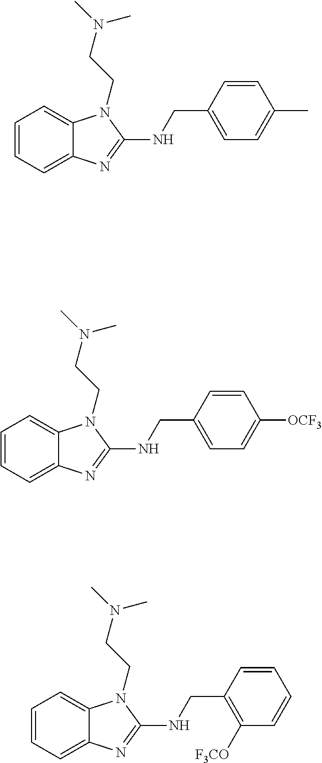 [SMILES: Cc1ccc(CNc2nc3ccccc3n2CCN(C)C)cc1 ; CN(C)CCn1c(NCc2ccc(OC(F)(F)F)cc2)nc2ccccc21 ; CN(C)CCn1c(NCc2ccccc2OC(F)(F)F)nc2ccccc21]

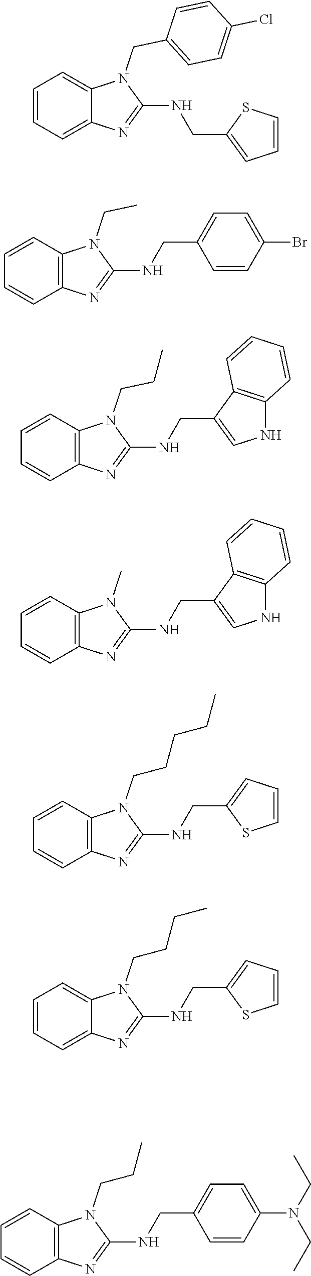

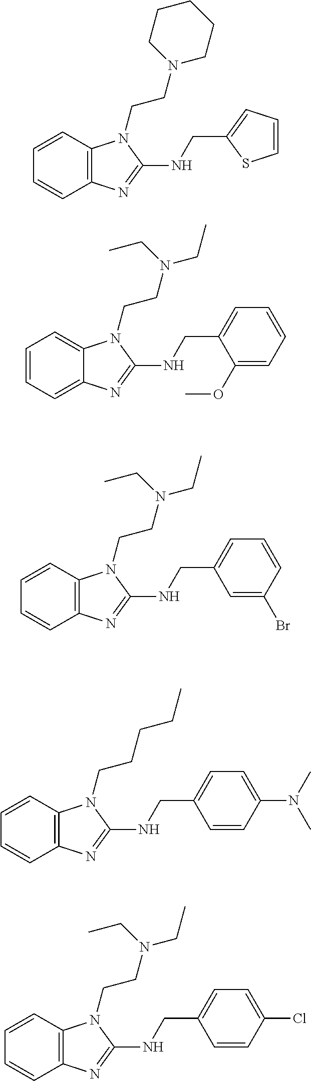 [SMILES: c1csc(CNc2nc3ccccc3n2CCN2CCCCC2)c1 ; CCN(CC)CCn1c(NCc2ccccc2OC)nc2ccccc21 ; CCN(CC)CCn1c(NCc2cccc(Br)c2)nc2ccccc21 ; CCCCCn1c(NCc2ccc(N(C)C)cc2)nc2ccccc21 ; CCN(CC)CCn1c(NCc2ccc(Cl)cc2)nc2ccccc21]

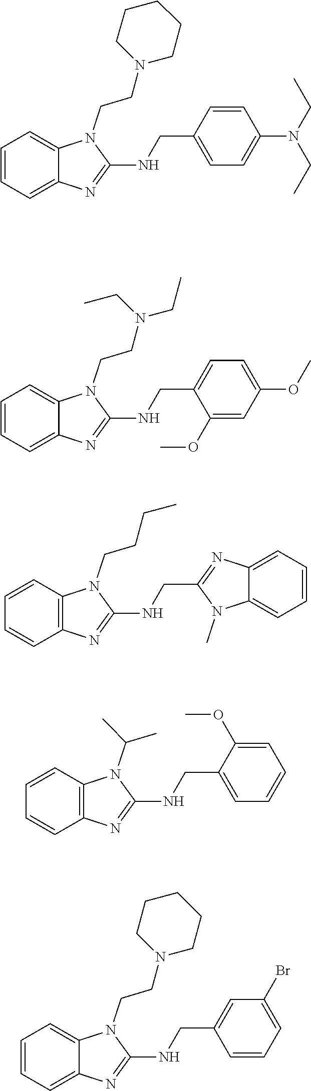 [SMILES: CCN(CC)c1ccc(CNc2nc3ccccc3n2CCN2CCCCC2)cc1 ; CCN(CC)CCn1c(NCc2ccc(OC)cc2OC)nc2ccccc21 ; CCCCn1c(NCc2nc3ccccc3n2C)nc2ccccc21 ; COc1ccccc1CNc1nc2ccccc2n1C(C)C ; Brc1cccc(CNc2nc3ccccc3n2CCN2CCCCC2)c1]

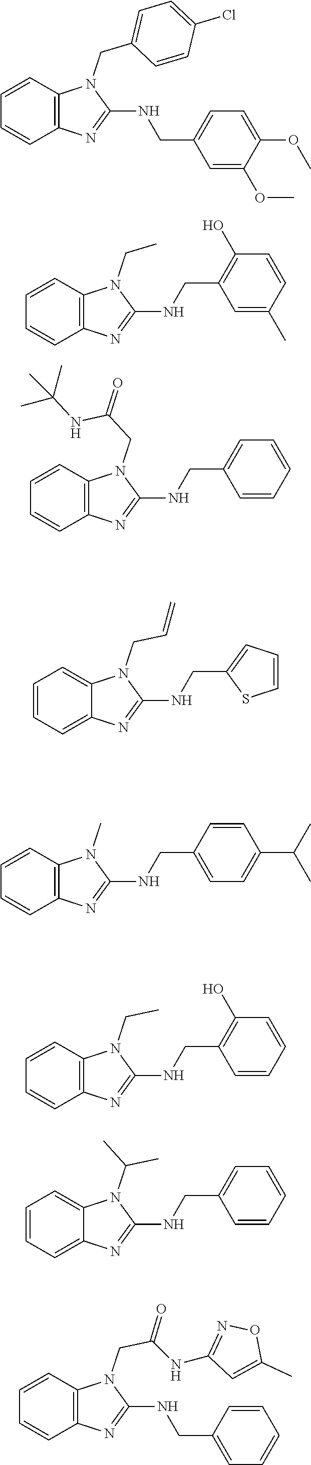

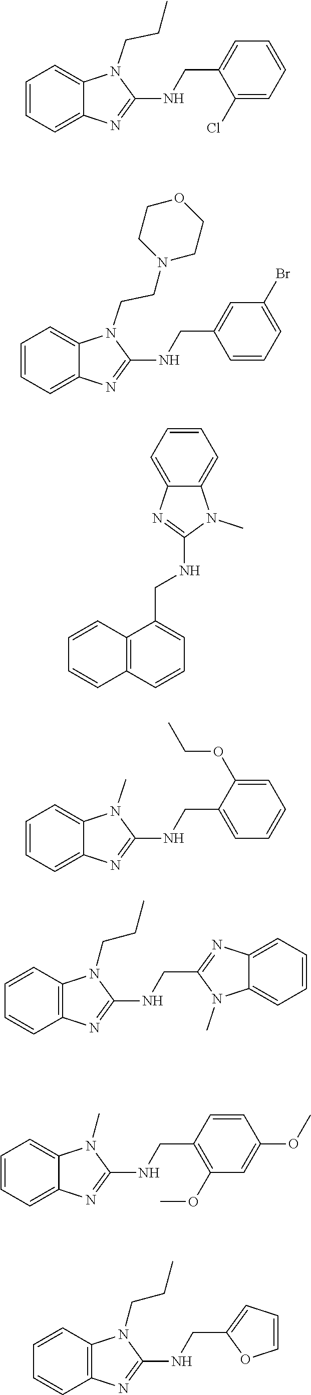

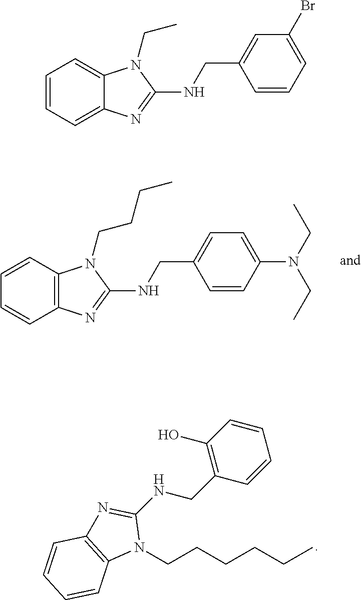 [SMILES: CCn1c(NCc2cccc(Br)c2)nc2ccccc21 ; CCCCn1c(NCc2ccc(N(CC)CC)cc2)nc2ccccc21 ; CCCCCCn1c(NCc2ccccc2O)nc2ccccc21]

In some embodiments, the compound is selected from the group consisting of:

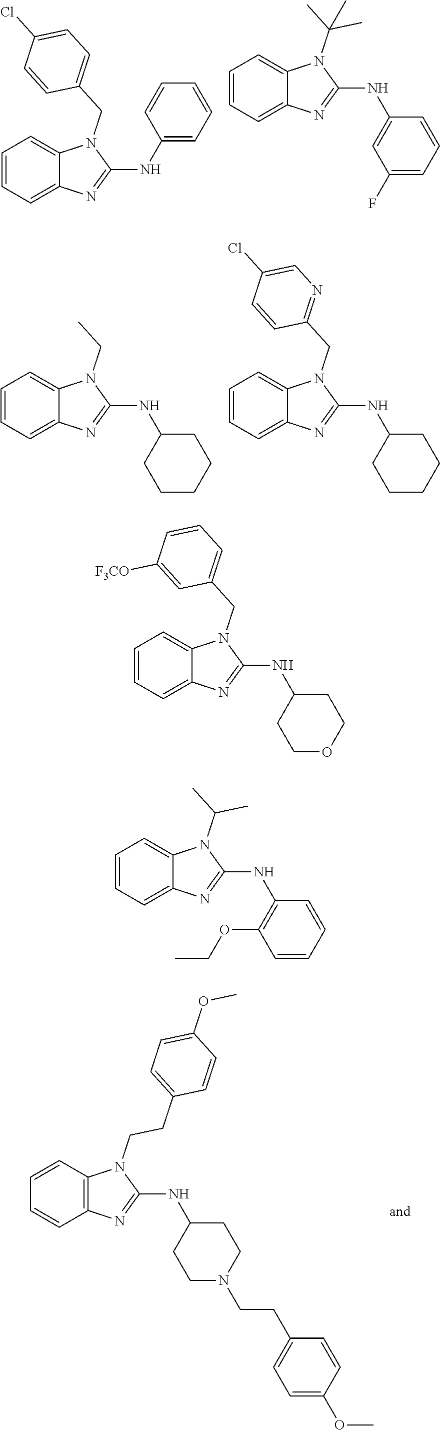

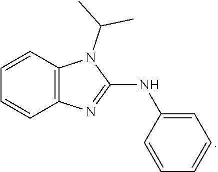 [SMILES: CC(C)n1c(Nc2ccccc2)nc2ccccc21]

In some embodiments, the compound is selected from the group consisting of:

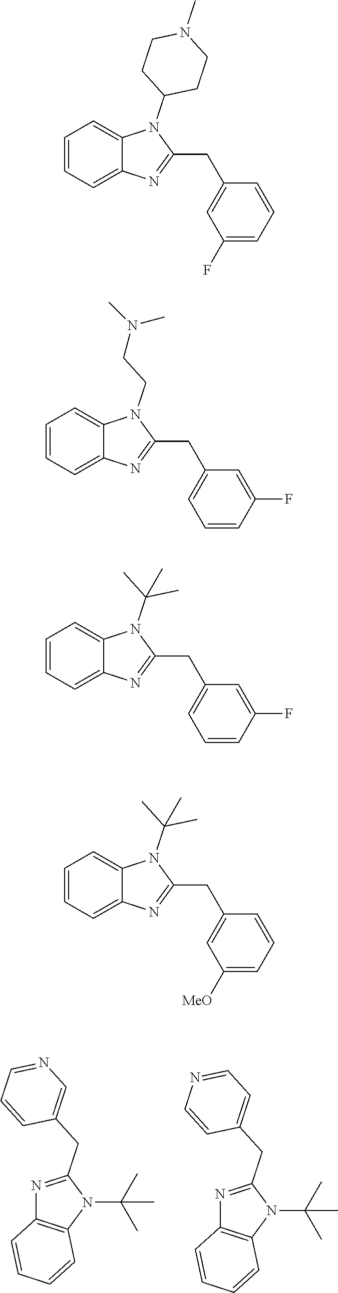 [SMILES: CN1CCC(n2c(Cc3cccc(F)c3)nc3ccccc32)CC1 ; CN(C)CCn1c(Cc2cccc(F)c2)nc2ccccc21 ; CC(C)(C)n1c(Cc2cccc(F)c2)nc2ccccc21 ; COc1cccc(Cc2nc3ccccc3n2C(C)(C)C)c1 ; CC(C)(C)n1c(Cc2cccnc2)nc2ccccc21 ; CC(C)(C)n1c(Cc2ccncc2)nc2ccccc21]

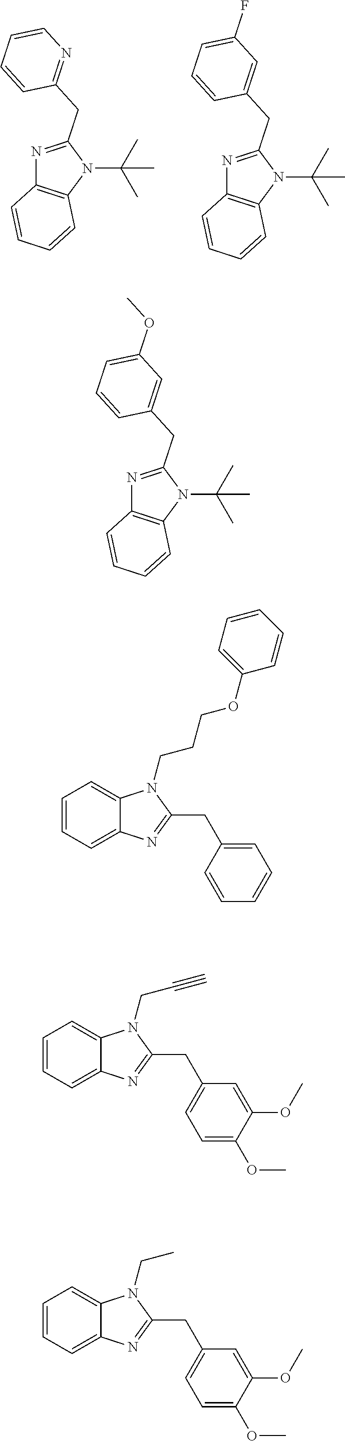 [SMILES: CC(C)(C)n1c(Cc2ccccn2)nc2ccccc21 ; CC(C)(C)n1c(Cc2cccc(F)c2)nc2ccccc21 ; COc1cccc(Cc2nc3ccccc3n2C(C)(C)C)c1 ; c1ccc(Cc2nc3ccccc3n2CCCOc2ccccc2)cc1 ; C#CCn1c(Cc2ccc(OC)c(OC)c2)nc2ccccc21 ; CCn1c(Cc2ccc(OC)c(OC)c2)nc2ccccc21]

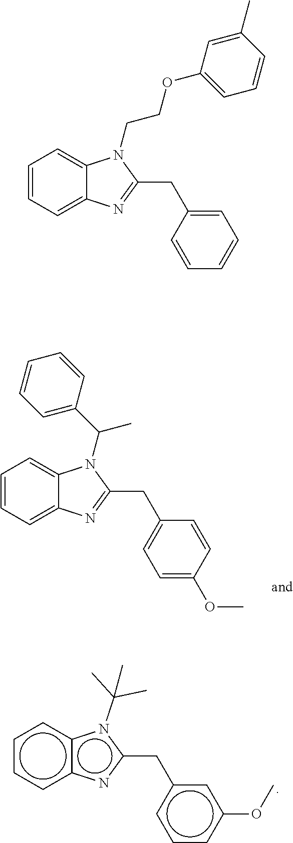

In some embodiments, the compound is selected from the group consisting of:

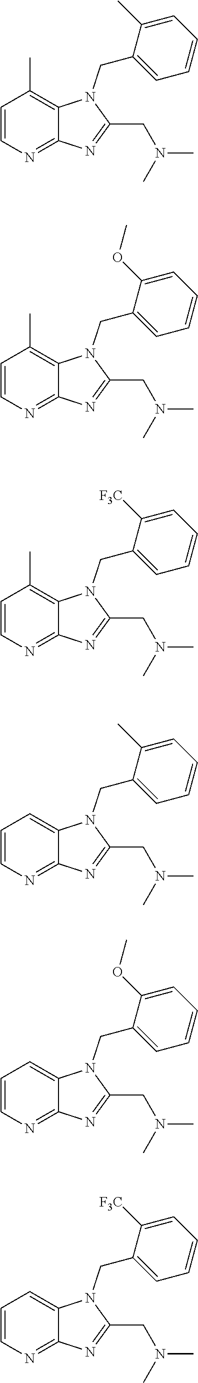

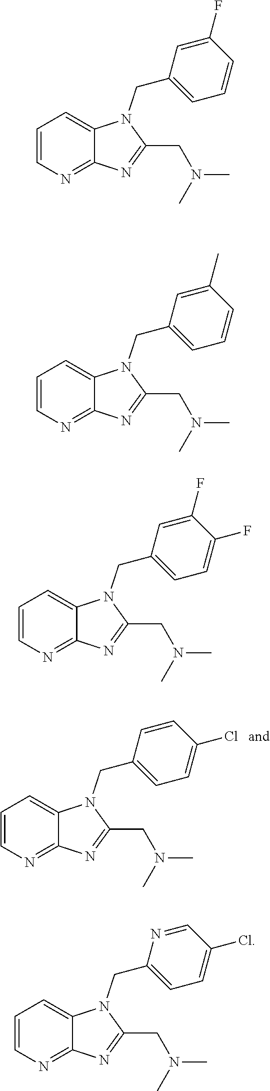

In some embodiments, the compound is selected from the group consisting of:

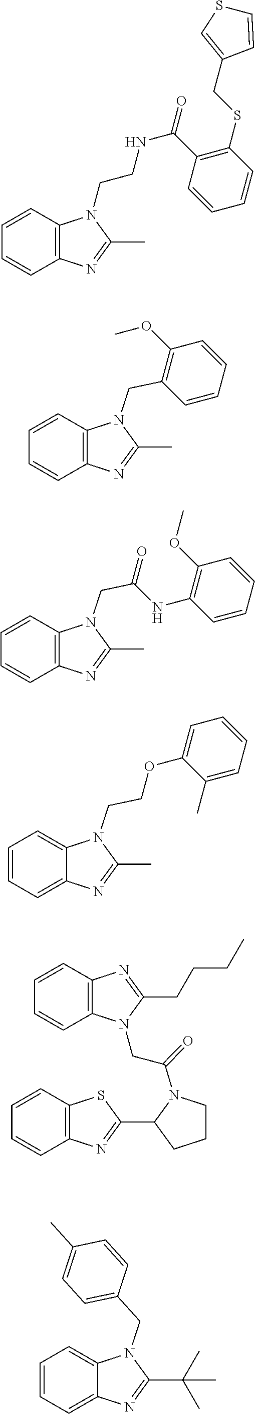 [SMILES: Cc1nc2ccccc2n1CCNC(=O)c1ccccc1SCc1ccsc1 ; COc1ccccc1Cn1c(C)nc2ccccc21 ; COc1ccccc1NC(=O)Cn1c(C)nc2ccccc21 ; Cc1ccccc1OCCn1c(C)nc2ccccc21 ; CCCCc1nc2ccccc2n1CC(=O)N1CCCC1c1nc2ccccc2s1 ; Cc1ccc(Cn2c(C(C)(C)C)nc3ccccc32)cc1]

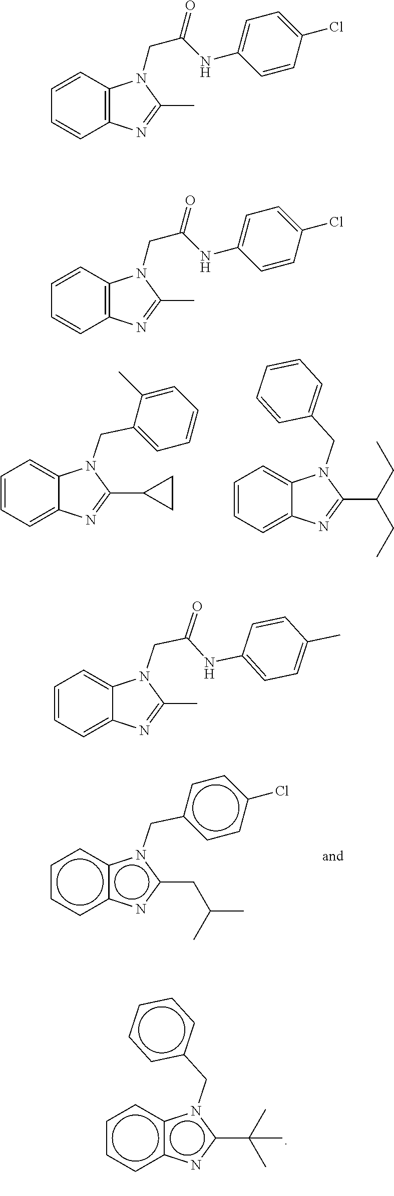

In some embodiments, the compound is selected from the group consisting of:

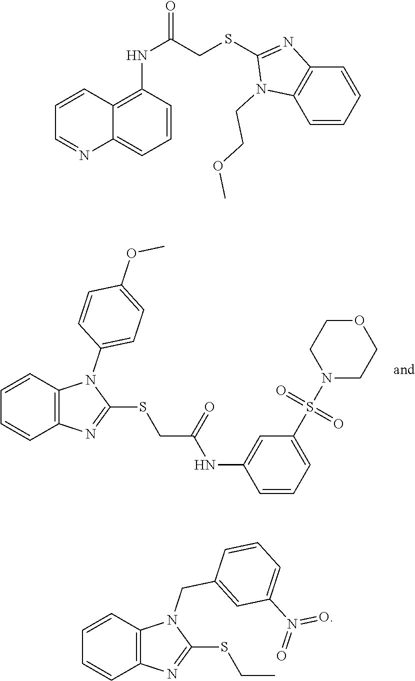 [SMILES: COCCn1c(SCC(=O)Nc2cccc3ncccc23)nc2ccccc21 ; COc1ccc(-n2c(SCC(=O)Nc3cccc(S(=O)(=O)N4CCOCC4)c3)nc3ccccc32)cc1 ; CCSc1nc2ccccc2n1Cc1cccc([N+](=O)[O-])c1]

In some embodiments, the compound is selected from the group consisting of:

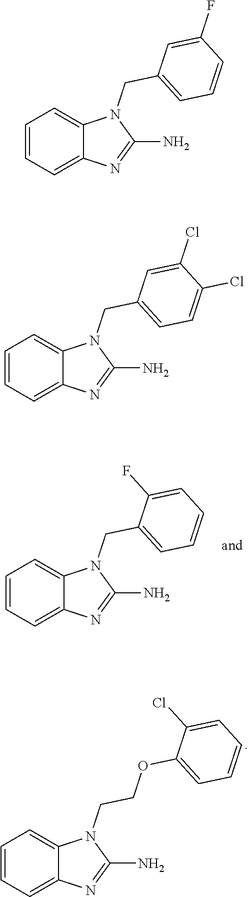 [SMILES: Nc1nc2ccccc2n1Cc1cccc(F)c1 ; Nc1nc2ccccc2n1Cc1ccc(Cl)c(Cl)c1 ; Nc1nc2ccccc2n1Cc1ccccc1F ; Nc1nc2ccccc2n1CCOc1ccccc1Cl]

In some embodiments, the compound is selected from the group consisting of:

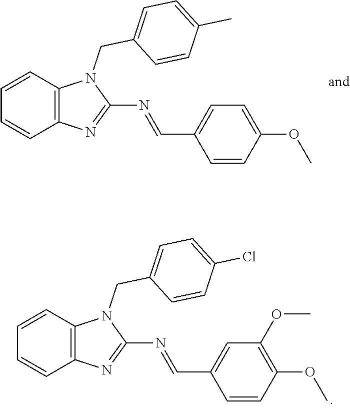 [SMILES: COc1ccc(/C=N/c2nc3ccccc3n2Cc2ccc(C)cc2)cc1 ; COc1ccc(/C=N/c2nc3ccccc3n2Cc2ccc(Cl)cc2)cc1OC]

In some embodiments, the compound is selected from the group consisting of:

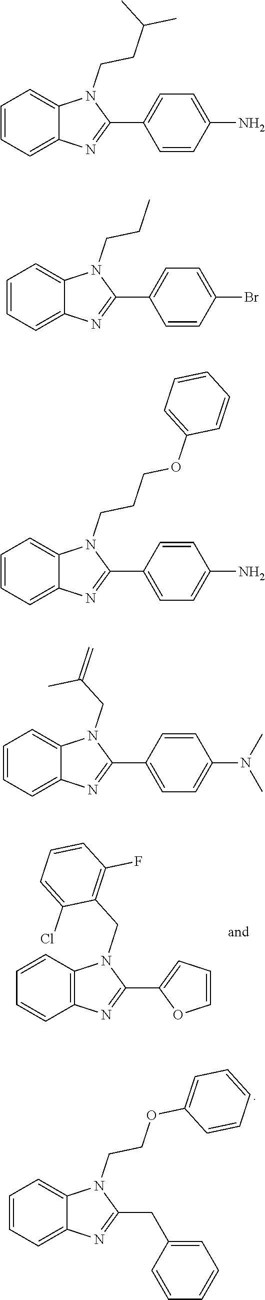 [SMILES: CC(C)CCn1c(-c2ccc(N)cc2)nc2ccccc21 ; CCCn1c(-c2ccc(Br)cc2)nc2ccccc21 ; Nc1ccc(-c2nc3ccccc3n2CCCOc2ccccc2)cc1 ; C=C(C)Cn1c(-c2ccc(N(C)C)cc2)nc2ccccc21 ; Fc1cccc(Cl)c1Cn1c(-c2ccco2)nc2ccccc21 ; c1ccc(Cc2nc3ccccc3n2CCOc2ccccc2)cc1]

In some embodiments, the compound is selected from the group consisting of:

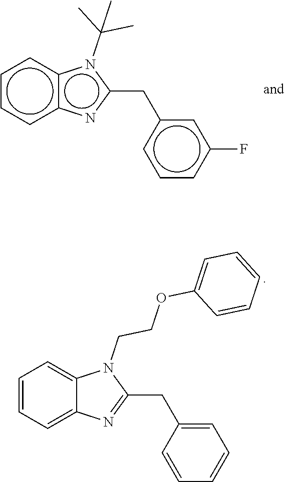

In some embodiments, the compound is selected from the group consisting of:

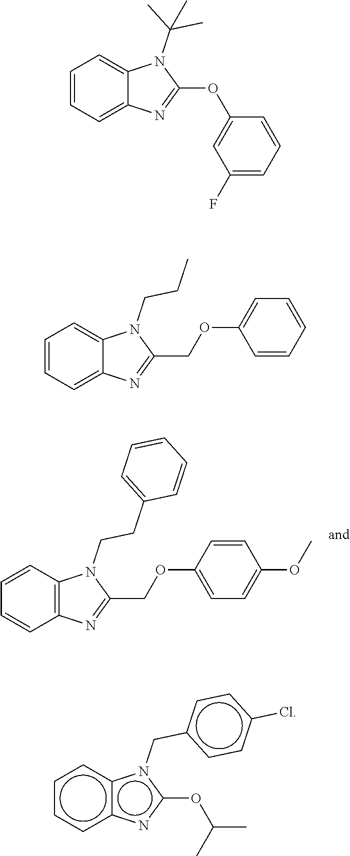

In some embodiments, the compound is selected from the group consisting of:

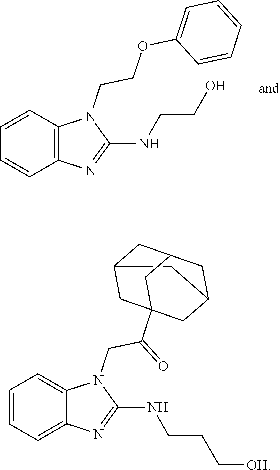 [SMILES: OCCNc1nc2ccccc2n1CCOc1ccccc1 ; O=C(Cn1c(NCCCO)nc2ccccc21)C12CC3CC(CC(C3)C1)C2]

In some embodiments, the compound is selected from the group consisting of:

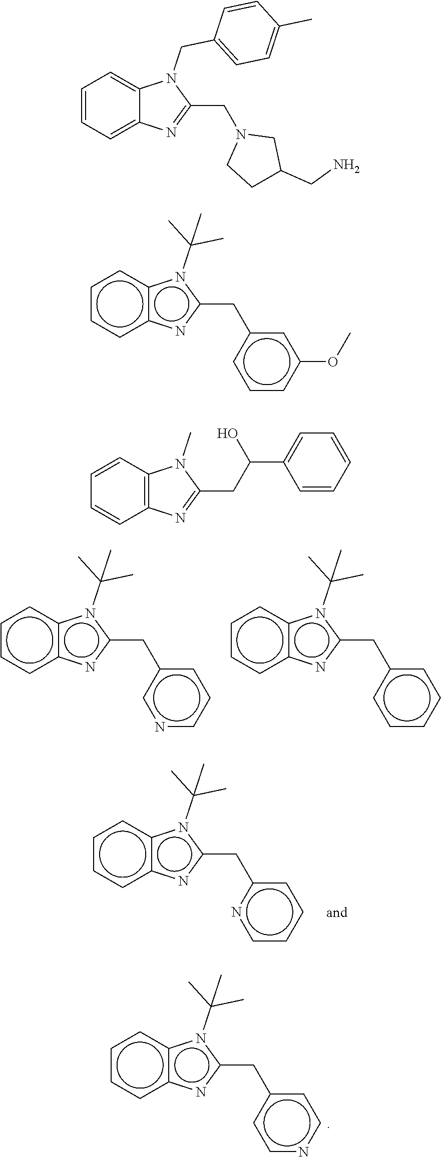

In some embodiments, the compound is selected from the group consisting of:

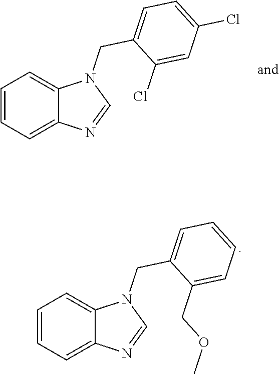 [SMILES: Clc1ccc(Cn2cnc3ccccc32)c(Cl)c1 ; COCc1ccccc1Cn1cnc2ccccc21]

In some embodiments, the compound is selected from the group consisting of:

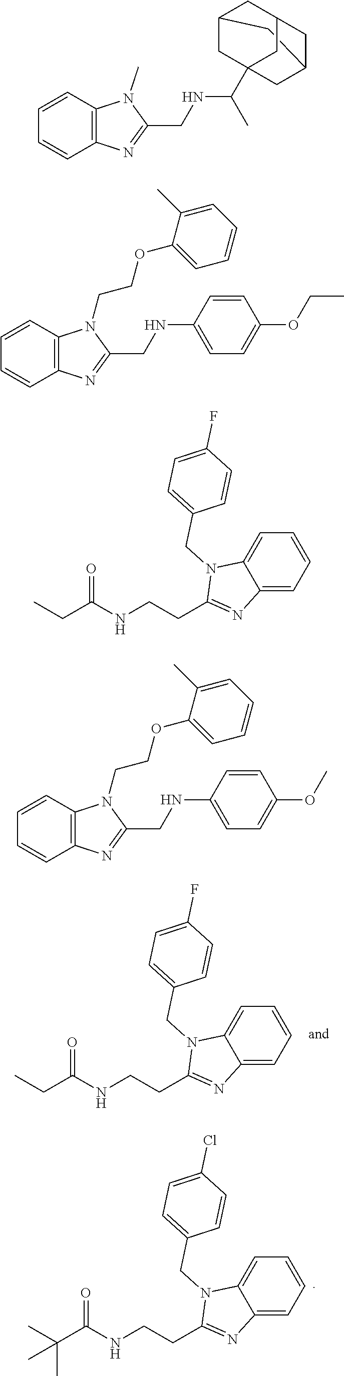

In certain embodiments, a compound of the invention may be racemic. In certain embodiments, a compound of the invention may be enriched in one enantiomer. For example, a compound of the invention may have greater than 30% ee, 40% ee, 50% ee, 60% ee, 70% ee, 80% ee, 90% ee, or even 95% or greater ee.

The compounds of the invention may have more than one stereocenter. Accordingly, the compounds of the invention may be enriched in one or more diastereomers. For example, a compound of the invention may have greater than 30% de, 40% de, 50% de, 60% de, 70% de, 80% de, 90% de, or even 95% or greater de. In certain embodiments, the compounds of the invention have substantially one isomeric configuration at one or more stereogenic centers, and have multiple isomeric configurations at the remaining stereogenic centers.

In certain embodiments, the enantiomeric excess of the stereocenter is at least 40% ee, 50% ee, 60% ee, 70% ee, 80% ee, 90% ee, 92% ee, 94% ee, 95% ee, 96% ee, 98% ee or greater ee.

As used herein, single bonds drawn without stereochemistry do not indicate the stereochemistry of the compound.

### Methods of Treatment

The non-selective Ca²⁺ permeable Transient Receptor Potential (TRP) channels act as sensors that transduce extracellular cues to the intracellular environment in diverse cellular processes, including actin remodeling and cell migration (Greka et al., Nat Neurosci 6, 837-845, 2003; Ramsey et al., Annu Rev Physiol 68, 619-647, 2006; Montell, Pflugers Arch 451, 19-28, 2005; Clapham, Nature 426, 517-524, 2003). Dynamic rearrangement of the actin cytoskeleton relies on spatiotemporally regulated Ca²⁺ influx (Zheng and Poo, Annu Rev Cell Dev Biol 23, 375-404, 2007); Brandman and Meyer, Science 322, 390-395, 2008); Collins and Meyer, Dev Cell 16, 160-161, 2009) and the small GTPases RhoA and Rac1 serve as key modulators of these changes (Etienne-Manneville and Hall, Nature 420, 629-635, 2002); Raftopoulou and Hall, Dev Biol 265, 23-32, 2004). RhoA induces stress fiber and focal adhesion formation, while Rac1 mediates lamellipodia formation (Etienne-Manneville and Hall, Nature 420, 629-635, 2002). The Transient Receptor Potential Cation Channel, subfamily C, member 5 (TRPC5) acts in concert with TRPC6 to regulate Ca2+ influx, actin remodeling, and cell motility in kidney podocytes and fibroblasts. TRPC5-mediated Ca²⁺ influx increases Rac1 activity, whereas TRPC6-mediated Ca²⁺ influx promotes RhoA activity. Gene silencing of TRPC6 channels abolishes stress fibers and diminishes focal contacts, rendering a motile, migratory cell phenotype. In contrast, gene silencing of TRPC5 channels rescues stress fiber formation, rendering a contractile cell phenotype. The results described herein unveil a conserved signaling mechanism whereby TRPC5 and TRPC6 channels control a tightly regulated balance of cytoskeletal dynamics through differential coupling to Rac1 and RhoA.

Ca²⁺-dependent remodeling of the actin cytoskeleton is a dynamic process that drives cell migration (Wei et al., Nature 457, 901-905, 2009). RhoA and Rac1 act as switches responsible for cytoskeletal rearrangements in migrating cells (Etienne-Manneville and Hall, Nature 420, 629-635, 2002); Raftopoulou and Hall, Dev Biol 265, 23-32, 2004). Activation of Rac1 mediates a motile cell phenotype, whereas RhoA activity promotes a contractile phenotype (Etienne-Manneville and Hall, Nature 420, 629-635, 2002). Ca²⁺ plays a central role in small GTPase regulation (Aspenstrom et al., Biochem J 377, 327-337, 2004). Spatially and temporally restricted flickers of Ca²⁺ are enriched near the leading edge of migrating cells (Wei et al., Nature 457, 901-905, 2009). Ca²⁺ microdomains have thus joined local bursts in Rac1 activity (Gardiner et al., Curr Biol 12, 2029-2034, 2002; Machacek et al., Nature 461, 99-103, 2009) as critical events at the leading edge. To date, the sources of Ca²⁺ influx responsible for GTPase regulation remain largely elusive. TRP (Transient Receptor Potential) channels generate time and space-limited Ca²⁺ signals linked to cell migration in fibroblasts and neuronal growth cones0. Specifically, TRPC5 channels are known regulators of neuronal growth cone guidance1 and their activity in neurons is dependent on PI3K and Rac1 activity (Bezzerides et al., Nat Cell Biol 6, 709-720, 2004).

Podocytes are neuronal-like cells that originate from the metanephric mesenchyme of the kidney glomerulus and are essential to the formation of the kidney filtration apparatus (Somlo and Mundel, Nat Genet. 24, 333-335, 2000; Fukasawa et al., J Am Soc Nephrol 20, 1491-1503, 2009). Podocytes possess an exquisitely refined repertoire of cytoskeletal adaptations to environmental cues (Somlo and Mundel, Nat Genet 24, 333-335, 2000, Garg et al., Mol Cell Biol 27, 8698-8712, 2007; Verma et al., J Clin Invest 116, 1346-1359, 2006; Verma et al., J Biol Chem 278, 20716-20723, 2003; Barletta et al., J Biol Chem 278, 19266-19271, 2003; Holzman et al., Kidney Int 56, 1481-1491, 1999; Ahola et al., Am J Pathol 155, 907-913, 1999; Tryggvason and Wartiovaara, N Engl J Med 354, 1387-1401, 2006; Schnabel and Farquhar, J Cell Biol 111, 1255-1263, 1990; Kurihara et al., Proc Natl Acad Sci USA 89, 7075-7079, 1992). Early events of podocyte injury are characterized by dysregulation of the actin cytoskeleton (Faul et al., Trends Cell Biol 17, 428-437, 2007; Takeda et al., J Clin Invest 108, 289-301, 2001; Asanuma et al., Nat Cell Biol 8, 485-491, 2006) and Ca²⁺ homeostasis (Hunt et al., J Am Soc Nephrol 16, 1593-1602, 2005; Faul et al., Nat Med 14, 931-938, 2008). These changes are associated with the onset of proteinuria, the loss of albumin into the urinary space, and ultimately kidney failure (Tryggvason and Wartiovaara, N Engl J Med 354, 1387-1401, 2006). The vasoactive hormone Angiotensin II induces Ca²⁺ influx in podocytes, and prolonged treatment results in loss of stress fibers (Hsu et al., J Mol Med 86, 1379-1394, 2008). While there is a recognized link between Ca²⁺ influx and cytoskeletal reorganization, the mechanisms by which the podocyte senses and transduces extracellular cues that modulate cell shape and motility remain elusive. TRP Canonical 6 (TRPC6) channel mutations have been linked to podocyte injury (Winn et al., Science 308, 1801-1804, 2005; Reiser et al., Nat Genet 37, 739-744, 2005; Moller et al., J Am Soc Nephrol 18, 29-36, 2007; Hsu et al., Biochim Biophys Acta 1772, 928-936, 2007), but little is known about the specific pathways that regulate this process. Moreover, TRPC6 shares close homology with six other members of the TRPC channel family (Ramsey et al., Annu Rev Physiol 68, 619-647, 2006; Clapham, Nature 426, 517-524, 2003). TRPC5 channels antagonize TRPC6 channel activity to control a tightly regulated balance of cytoskeletal dynamics through differential coupling to distinct small GTPases.

Proteinuria

Proteinuria is a pathological condition wherein protein is present in the urine. Albuminuria is a type of proteinuria. Microalbuminuria occurs when the kidney leaks small amounts of albumin into the urine. In a properly functioning body, albumin is not normally present in urine because it is retained in the bloodstream by the kidneys. Microalbuminuria is diagnosed either from a 24-hour urine collection (20 to 200 μg/min) or, more commonly, from elevated concentrations (30 to 300 mg/L) on at least two occasions. Microalbuminuria can be a forerunner of diabetic nephropathy. An albumin level above these values is called macroalbuminuria. Subjects with certain conditions, e.g., diabetic nephropathy, can progress from microalbuminuria to macroalbuminuria and reach a nephrotic range (>3.5 g/24 hours) as kidney disease reaches advanced stages.

Causes of Proteinuria

Proteinuria can be associated with a number of conditions, including focal segmental glomerulosclerosis, TgA nephropathy, diabetic nephropathy, lupus nephritis, membranoproliferative glomerulonephritis, progressive (crescentic) glomerulonephritis, and membranous glomerulonephritis.

A. Focal Segmental Glomerulosclerosis (FSGS)

Focal Segmental Glomerulosclerosis (FSGS) is a disease that attacks the kidney's filtering system (glomeruli) causing serious scarring. FSGS is one of the many causes of a disease known as Nephrotic Syndrome, which occurs when protein in the blood leaks into the urine (proteinuria).

Very few treatments are available for patients with FSGS. Many patients are treated with steroid regimens, most of which have very harsh side effects. Some patients have shown to respond positively to immunosuppressive drugs as well as blood pressure drugs which have shown to lower the level of protein in the urine. To date, there is no commonly accepted effective treatment or cure and there are no FDA approved drugs to treat FSGS. Therefore, more effective methods to reduce or inhibit proteinuria are desirable.

B. IgA Nephropathy

IgA nephropathy (also known as IgA nephritis, IgAN, Berger's disease, and synpharyngitic glomerulonephritis) is a form of glomerulonephritis (inflammation of the glomeruli of the kidney). IgA nephropathy is the most common glomerulonephritis throughout the world. Primary IgA nephropathy is characterized by deposition of the IgA antibody in the glomerulus. There are other diseases associated with glomerular IgA deposits, the most common being Henoch-Schnlein purpura (HSP), which is considered by many to be a systemic form of IgA nephropathy. Henoch-Schönlein purpura presents with a characteristic purpuric skin rash, arthritis, and abdominal pain and occurs more commonly in young adults (16-35 yrs old). HSP is associated with a more benign prognosis than IgA nephropathy. In IgA nephropathy there is a slow progression to chronic renal failure in 25-30% of cases during a period of 20 years.

C. Diabetic Nephropathy

Diabetic nephropathy, also known as Kimmelstiel-Wilson syndrome and intercapillary glomerulonephritis, is a progressive kidney disease caused by angiopathy of capillaries in the kidney glomeruli. It is characterized by nephrotic syndrome and diffuse glomerulosclerosis. It is due to longstanding diabetes mellitus and is a prime cause for dialysis. The earliest detectable change in the course of diabetic nephropathy is a thickening in the glomerulus. At this stage, the kidney may start allowing more serum albumin than normal in the urine. As diabetic nephropathy progresses, increasing numbers of glomeruli are destroyed by nodular glomerulosclerosis and the amount of albumin excreted in the urine increases.

D. Lupus Nephritis

Lupus nephritis is a kidney disorder that is a complication of systemic lupus erythematosus. Lupus nephritis occurs when antibodies and complement build up in the kidneys, causing inflammation. It often causes proteinuria and may progress rapidly to renal failure. Nitrogen waste products build up in the bloodstream. Systemic lupus erythematosus causes various disorders of the internal structures of the kidney, including interstitial nephritis. Lupus nephritis affects approximately 3 out of 10,000 people.

E. Membranoproliferative Glomerulonephritis I/II/III

Membranoproliferative glomerulonephritis is a type of glomerulonephritis caused by deposits in the kidney glomerular mesangium and basement membrane thickening, activating complement and damaging the glomeruli. There are three types of membranoproliferative glomerulonephritis. Type I is caused by immune complexes depositing in the kidney and is believed to be associated with the classical complement pathway. Type II is similar to Type I, however, it is believed to be associated with the alternative complement pathway. Type III is very rare and it is characterized by a mixture of subepithelial deposits and the typical pathological findings of Type I disease.

F. Progressive (Crescentic) Glomerulonephritis

Progressive (crescentic) glomerulonephritis (PG) is a syndrome of the kidney that, if left untreated, rapidly progresses into acute renal failure and death within months. In 50% of cases, PG is associated with an underlying disease such as Goodpasture's syndrome, systemic lupus erythematosus, or Wegener granulomatosis; the remaining cases are idiopathic. Regardless of the underlying cause, PG involves severe injury to the kidney's glomeruli, with many of the glomeruli containing characteristic crescent-shaped scars. Patients with PG have hematuria, proteinuria, and occasionally, hypertension and edema. The clinical picture is consistent with nephritic syndrome, although the degree of proteinuria may occasionally exceed 3 g/24 hours, a range associated with nephrotic syndrome. Untreated disease may progress to decreased urinary volume (oliguria), which is associated with poor kidney function.

G. Membranous Glomerulonephritis

Membranous glomerulonephritis (MGN) is a slowly progressive disease of the kidney affecting mostly patients between ages of 30 and 50 years, usually Caucasian. It can develop into nephrotic syndrome. MGN is caused by circulating immune complex. Current research indicates that the majority of the immune complexes are formed via binding of antibodies to antigens in situ to the glomerular basement membrane. The said antigens may be endogenous to the basement membrane, or deposited from systemic circulation.

Measurement of Urine Protein Levels

Protein levels in urine can be measured using methods known in the art. Until recently, an accurate protein measurement required a 24-hour urine collection. In a 24-hour collection, the patient urinates into a container, which is kept refrigerated between trips to the bathroom. The patient is instructed to begin collecting urine after the first trip to the bathroom in the morning. Every drop of urine for the rest of the day is to be collected in the container. The next morning, the patient adds the first urination after waking and the collection is complete.

More recently, researchers have found that a single urine sample can provide the needed information. In the newer technique, the amount of albumin in the urine sample is compared with the amount of creatinine, a waste product of normal muscle breakdown. The measurement is called a urine albumin-to-creatinine ratio (UACR). A urine sample containing more than 30 milligrams of albumin for each gram of creatinine (30 mg/g) is a warning that there may be a problem. If the laboratory test exceeds 30 mg/g, another UACR test should be performed 1 to 2 weeks later. If the second test also shows high levels of protein, the person has persistent proteinuria, a sign of declining kidney function, and should have additional tests to evaluate kidney function.

Tests that measure the amount of creatinine in the blood will also show whether a subject's kidneys are removing wastes efficiently. Too much creatinine in the blood is a sign that a person has kidney damage. A physician can use the creatinine measurement to estimate how efficiently the kidneys are filtering the blood. This calculation is called the estimated glomerular filtration rate, or eGFR. Chronic kidney disease is present when the eGFR is less than 60 milliliters per minute (mL/min).

TRPC5

TRPC is a family of transient receptor potential cation channels in animals. TRPC5 is subtype of the TRPC family of mammalian transient receptor potential ion channels. Three examples of TRPC5 are highlighted below in Table 1.

TABLE 1
The TRPC5 orthologs float three different species along
with their GenBank Ref Seq Accession Numbers.

| Species | Nucleic Acid | Amino Acid | Gene ID |
|---|---|---|---|
| Homo sapiens | NM_012471.2 | NP_036603.1 | 7224 |
| Mus musculus | NM_009428.2 | NP_033454.1 | 22067 |
| Rattus norvegicus | NM_080898.2 | NP_543174.1 | 140933 |

Accordingly, in certain embodiments, the invention provides methods for treating, or the reducing risk of developing, a kidney disease comprising administering to a subject in need thereof a therapeutically effective amount of a compound of the invention (e.g., a compound of Formula I), or a pharmaceutical composition comprising said compound.

In some embodiments, the kidney disease is selected from the group consisting of Focal Segmental Glomerulosclerosis (FSGS), Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive GN, anti-GBM disease, C3 glomerulonephritis, hypertensive nephrosclerosis, and IgA nephropathy. In some embodiments, the kidney disease is proteinuria. In some embodiments, the kidney disease is microalbuminuria or macroalbuminuria.

The invention also provides methods of treating, or the reducing risk of developing, anxiety, or depression, or cancer, comprising administering to a subject in need thereof a therapeutically effective amount of a compound of the invention (e.g., a compound of Formula I), or a pharmaceutical composition comprising said compound.

Subjects to be Treated

In one aspect of the invention, a subject is selected on the basis that they have, or are at risk of developing, a kidney disease, anxiety, depression, or cancer.

Subjects that have, or are at risk of developing, proteinuria include those with diabetes, hypertension, or certain family backgrounds. In the United States, diabetes is the leading cause of end-stage renal disease (ESRD). In both type 1 and type 2 diabetes, albumin in the urine is one of the first signs of deteriorating kidney function. As kidney function declines, the amount of albumin in the urine increases. Another risk factor for developing proteinuria is hypertension. Proteinuria in a person with high blood pressure is an indicator of declining kidney function. If the hypertension is not controlled, the person can progress to full kidney failure. African Americans are more likely than Caucasians to have high blood pressure and to develop kidney problems from it, even when their blood pressure is only mildly elevated. Other groups at risk for proteinuria are American Indians, Hispanics/Latinos, Pacific Islander Americans, older adults, and overweight subjects.

In one aspect of the invention, a subject is selected on the basis that they have, or are at risk of developing proteinuria. A subject that has, or is at risk of developing, proteinuria is one having one or more symptoms of the condition. Symptoms of proteinuria are known to those of skill in the art and include, without limitation, large amounts of protein in the urine, which may cause it to look foamy in the toilet. Loss of large amounts of protein may result in edema, where swelling in the hands, feet, abdomen, or face may occur. These are signs of large protein loss and indicate that kidney disease has progressed. Laboratory testing is the only way to find out whether protein is in a subject's urine before extensive kidney damage occurs.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the subject is a mammal. In some embodiments, the subject is a human.

### EXAMPLES

The invention is further described in the following examples, which do not limit the scope of the invention described in the claims.

### Example 1: Synthetic Methods

The following illustrate synthetic routes to exemplary compounds of the invention.

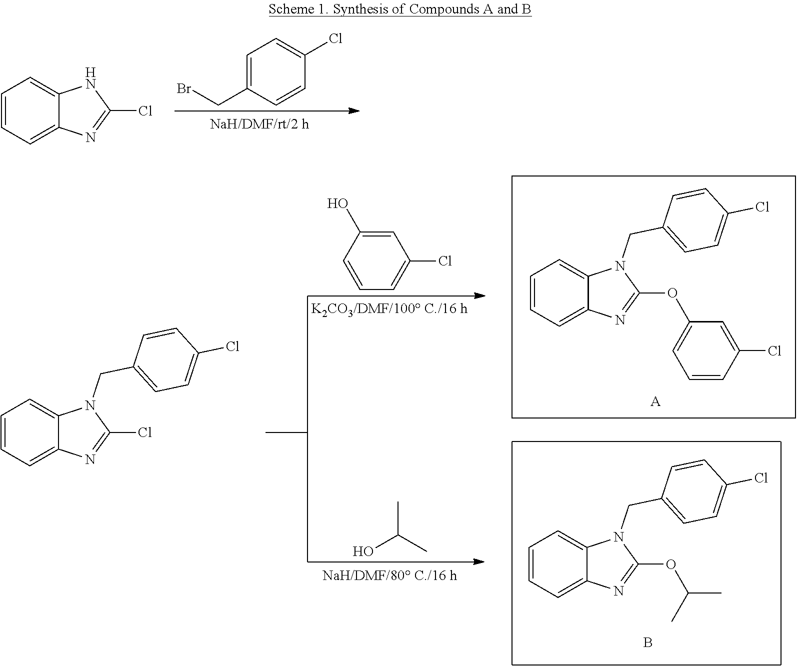

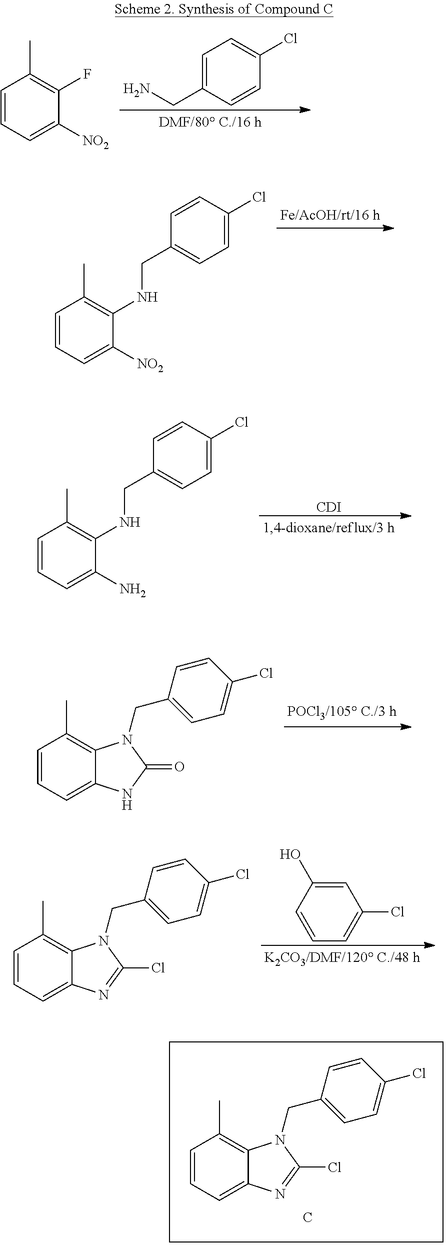

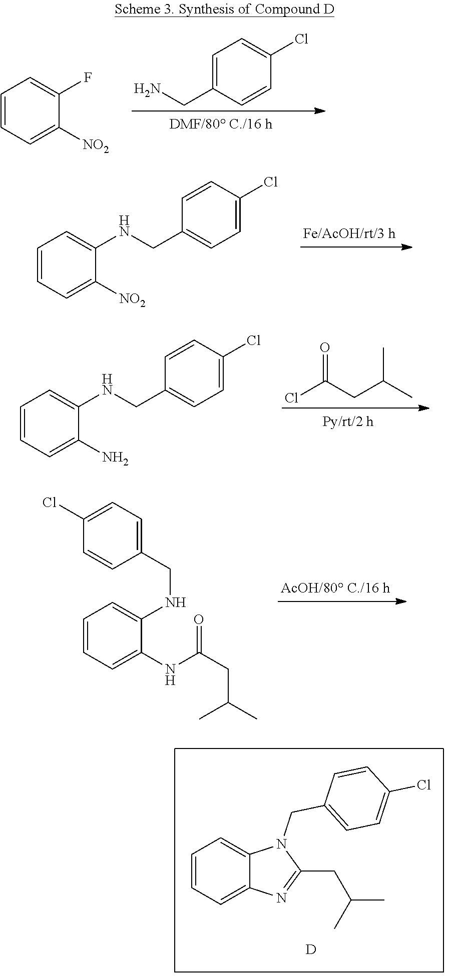

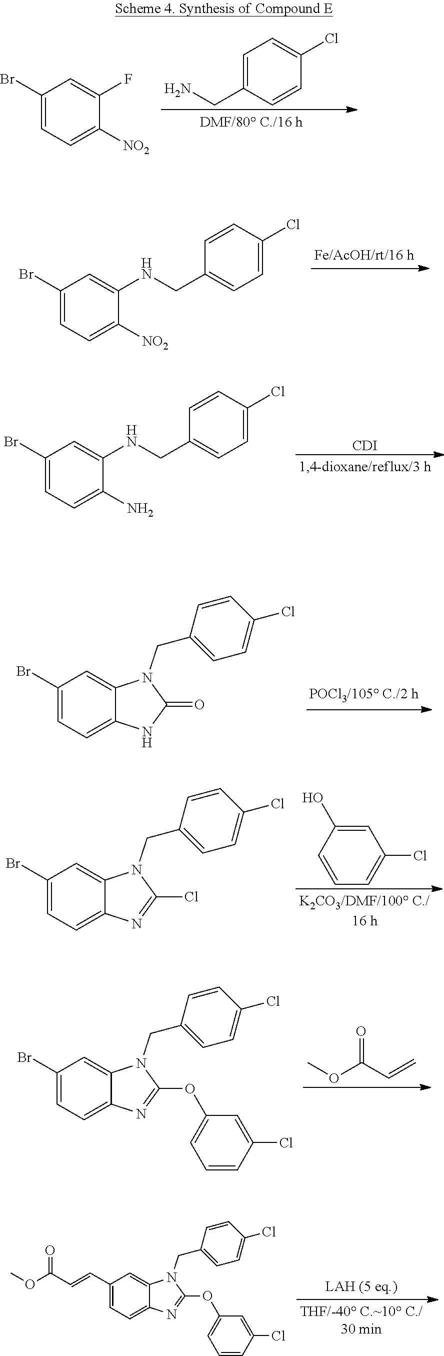

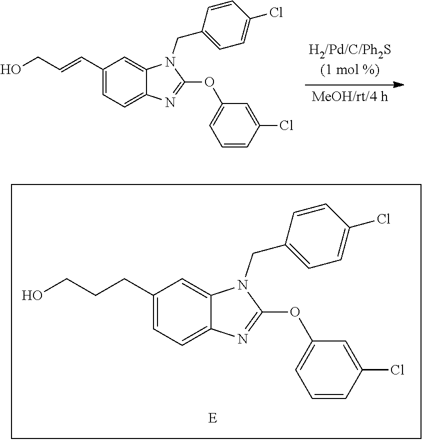 [SMILES: OC/C=C/c1ccc2nc(Oc3cccc(Cl)c3)n(Cc3ccc(Cl)cc3)c2c1 ; OCCCc1ccc2nc(Oc3cccc(Cl)c3)n(Cc3ccc(Cl)cc3)c2c1]

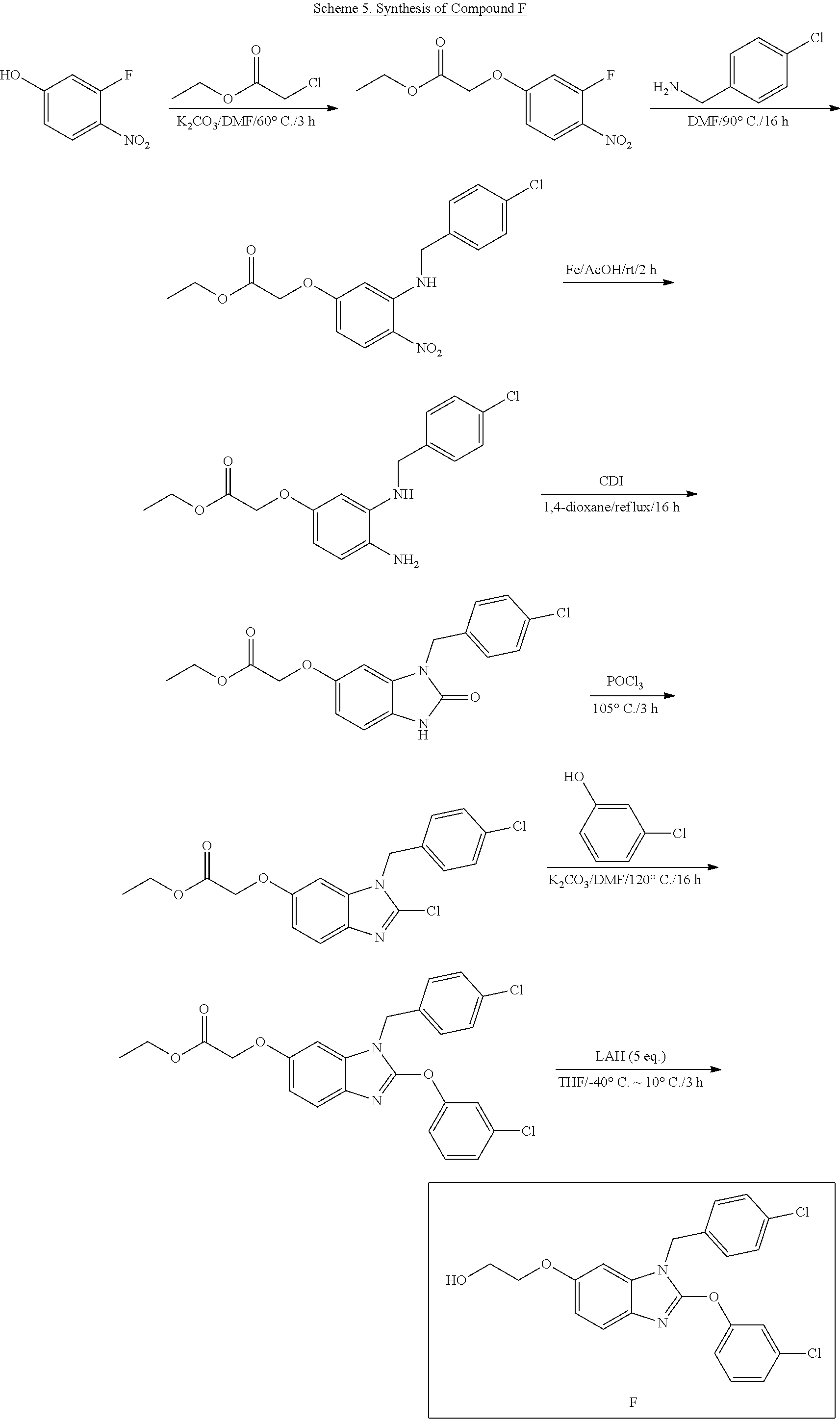

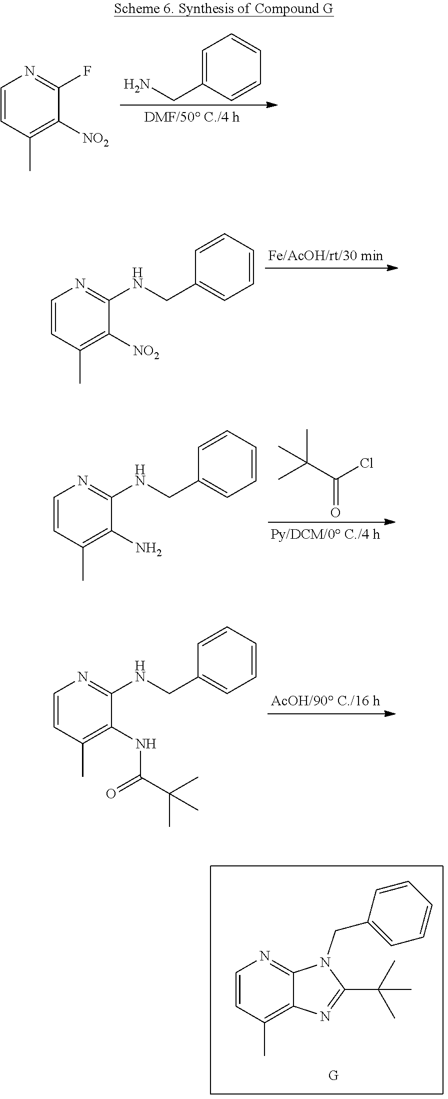

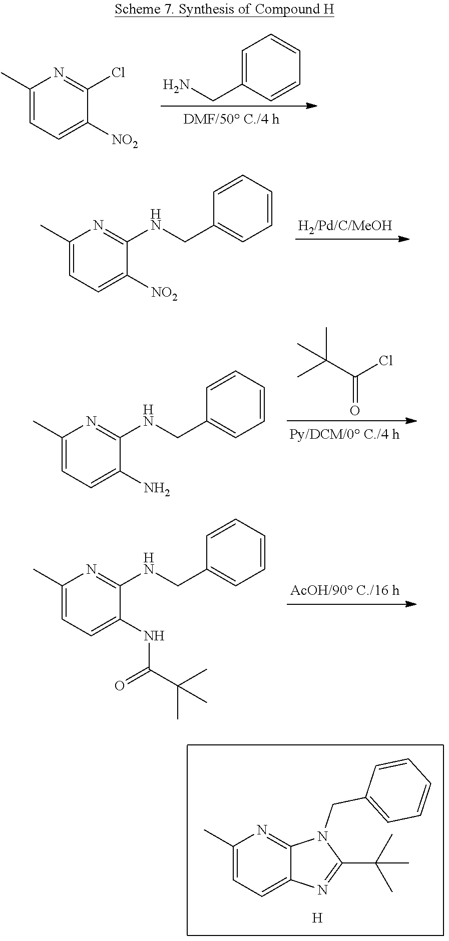

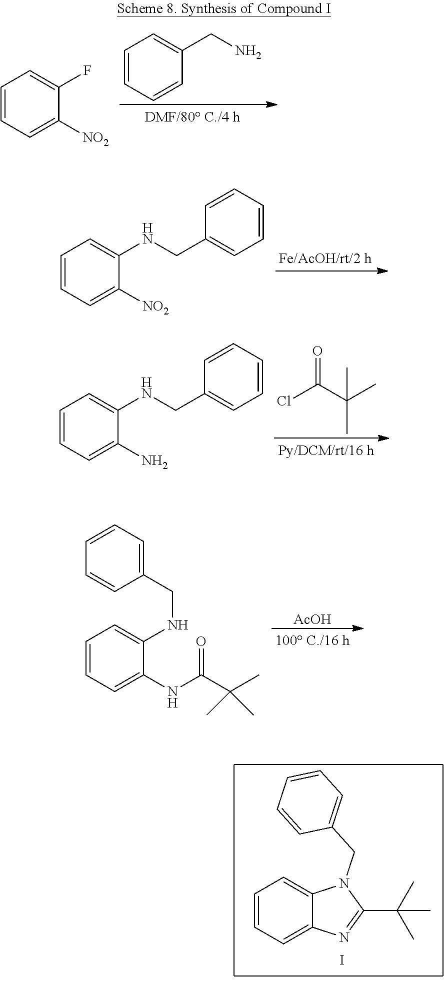

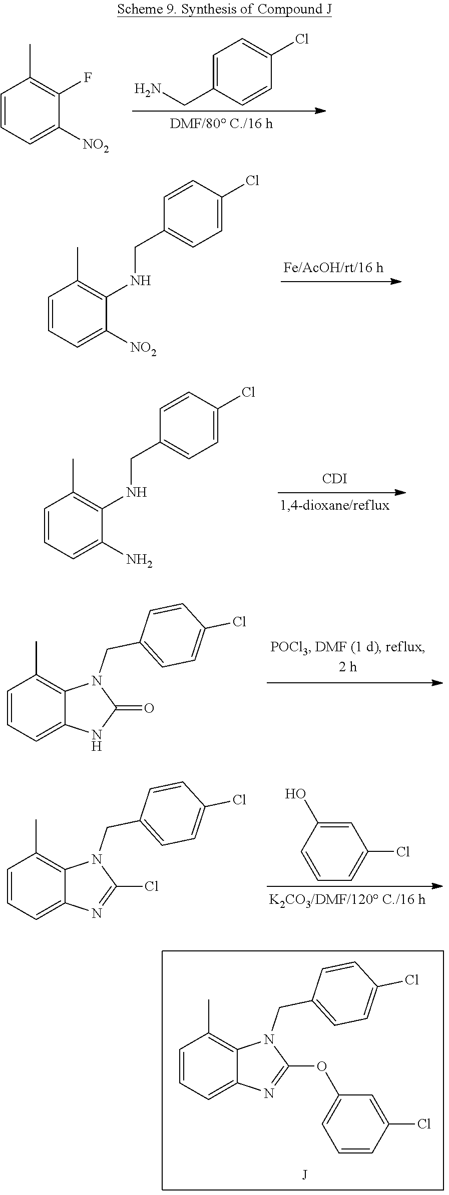

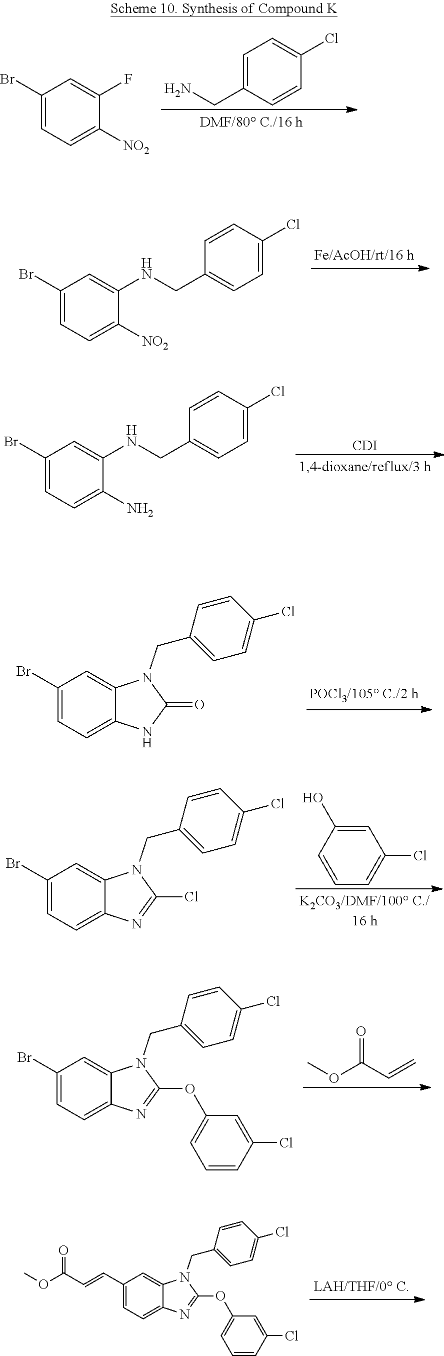

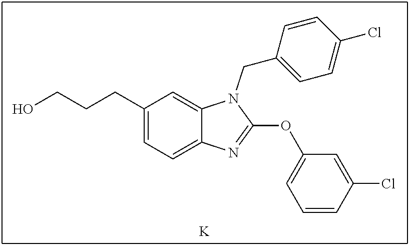

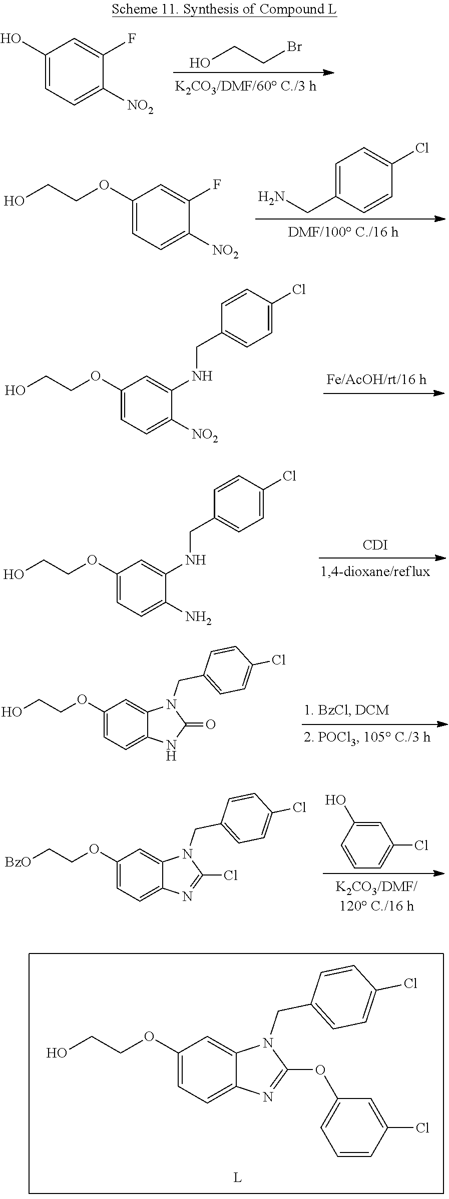

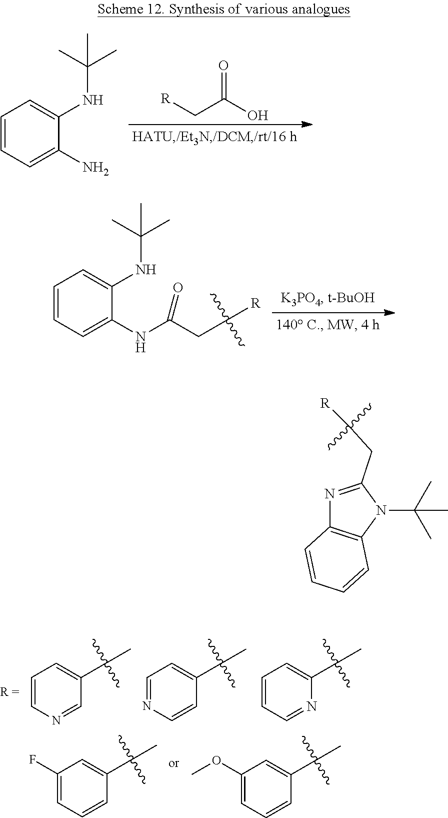

### Example 2: TRPCx Assay Protocols

I. Fluorescence-Based Assays

A. TRPC4

HEK 293 cells expressing human TRPC4 cells were trypsinised, counted and seeded in black, clear-bottomed 96-well plates at a density of 50,000 cells per well and incubated overnight. Next day, the cells were loaded with membrane potential dye. Dye solution was made up according to the manufacturer's instructions in HEPES buffered Hank's balanced salt solution (HBSS). Dye solution (10 μL) was added to the wells and incubated at 37° C. for 1 hour. The test compounds and standard inhibitors were added to the wells and incubated at room temperature for 10 minutes. The plates were then placed in the flexstation and fluorescence monitored every 1.52 seconds. After 20 seconds, 10 μL of the appropriate standard agonist was added and the fluorescence monitored for 2 minutes at ex/emm: 530 nm/565 nm

B. TRPC5

HEK 293 cells expressing human TRPC5 cells were trypsinised, counted and seeded in black, clear-bottomed 96-well plates at a density of 50,000 cells per well and incubated overnight. Next day, the cells were loaded with membrane potential dye. Dye solution was made up according to the manufacturer's instructions in HEPES buffered Hank's balanced salt solution (HBSS). Dye solution (10 μL) was added to the wells and incubated at 37° C. for 1 hour. The test compounds and standard inhibitors were added to the wells and incubated at room temperature for 10 minutes. The plates were then placed in the flexstation and fluorescence monitored every 1.52 seconds. After 20 seconds, 10 μL of the appropriate standard agonist was added and the fluorescence monitored for 2 minutes at ex/emm: 530 nm/565 nm.

C. TRPC6

HEK 293 cells expressing human TRPC6 cells were trypsinised, counted and seeded in black, clear-bottomed 96-well plates at a density of 50,000 cells per well and incubated overnight. Next day, the cells were loaded with membrane potential dye (Molecular Devices, cat: R8127). Dye solution was made up according to the manufacturer's instructions in HEPES buffered Hank's balanced salt solution (HBSS). Dye solution was added to the wells and incubated at 37° C. for 1 hour. The test compounds and standard inhibitors were added to the wells and incubated at room temperature for 10 minutes prior to addition of activator. The plates were then placed in the flexstation and fluorescence monitored every 1.52 seconds. After 20 seconds, the standard activator (Carbachol) was added and the fluorescence monitored for 2 minutes at ex/emm: 530 nm/565 nm.

II. Automated Patch Clamp Assay (Qpatch)

A. TRPC5

HEK 293 cells expressing human TRPC5 were harvested, re-suspended in serum free medium, and added to the automated platform and used within 2-3 hours. Internal and external physiological solutions were freshly prepared prior to the assay. The external solution contained: 145 mM NaCl, 4 mM KCl, 2 mM CaCl2), 1 mM MgCl2, 10 mM HEPES, 10 mM glucose, pH 7.4 with NaOH and 300 mOsm/L. The internal solution contained 120 mM L-aspartic acid, 120 mM CsOH.H2O, 20 mM CsCl, 2 mM MgCl2, 8.8 mM CaCl2), 10 mM EGTA, 10 mM HEPES, 10 mM Glucose, 0.1 mM GTP and 2 mM Na2ATP; pH 7.2 with CsOH and 290 mOsm/L. The free internal Ca2+ concentration was buffered to 1 M.

The automated electrophysiological platform QPatch 16 from Sophion (Denmark) was used to carry out the compounds profiling. The series resistance and quality of seals were continuously monitored during the experiments. Data was analyzed using Sophion QPatch assay software 5.6 (Odense). IC50 values were calculated using a least squares regression algorithm (Hill equation).

To monitor the ion currents, a voltage ramp from −100 mV to +100 mV, over 300 ms, was applied every 10 seconds, from a holding potential of −60 mV.

After recording for a minimum of 60 seconds control period, Rosiglitazone (30 uM), was applied to activate the channel.

B. TRPC4

HEK 293 cells expressing human TRPC4 were harvested, re-suspended in serum free medium, added to the automated platform and used within 2-3 hours. Internal and external physiological solutions were freshly prepared prior the assay. The external solution contained: 145 mM NaCl, 4 mM KCl, 2 mM CaCl2), 1 mM MgCl2, 10 mM HEPES, 10 mM glucose, pH 7.4 with NaOH and 300 mOsm/L. The internal solution contained 120 mM L-aspartic acid, 120 mM CsOH.H2O, 20 mM CsCl, 2 mM MgCl2, 10 mM EGTA, 10 mM HEPES, 10 mM Glucose, and 2 mM Na2ATP; pH 7.2 with CsOH and 290 mOsm/L.

The TRPC4 channel agonist Englerin was used to activate and assess test compounds.

The automated electrophysiological platform Qpatch 16 from Sophion (Denmark) was used to carry out the compounds profiling. The series resistance and quality of seals were continuously monitored during the experiments. Data was analyzed using Sophion Qpatch assay software 5.6 (Odense) and Microsoft Office Excel 2007. IC50 values were calculated using a least squares regression algorithm (Hill equation).

C. TRPC6

HEK 293 cells expressing human TRPC6 were harvested, re-suspended in serum free medium, added to the automated platform and used within 2-3 hours. Internal and external physiological solutions were freshly prepared prior the assay. The external solution contained: 145 mM NaCl, 4 mM KCl, 2 mM CaCl2), 1 mM MgCl2, 10 mM HEPES, 10 mM glucose, pH 7.4 with NaOH and 300 mOsm/L. The internal solution contained 120 mM L-aspartic acid, 120 mM CsOH.H2O, 20 mM CsCl, 2 mM MgCl2, 10 mM EGTA, 10 mM HEPES, 10 mM Glucose, and 2 mM Na2ATP; pH 7.2 with CsOH and 290 mOsm/L.

The agonist OAG EC50 was used to activate TRPC6 and assess test compounds.

The automated electrophysiological platform Qpatch 16 from Sophion (Denmark) was used to carry out the compounds profiling. The series resistance and quality of seals were continuously monitored during the experiments. Data was analyzed using Sophion Qpatch assay software 5.6 (Odense) and Microsoft Office Excel 2007. IC50 values were calculated using a least squares regression algorithm (Hill equation).

### Example 3: Exemplary Biological Assay Data

TABLE 2
Percent inhibition of TRPC5 or TRPC6 by representative compounds of the
disclosure, measured utilizing a Ca²⁺ flux assay in FLIPR format (see above) following treatment
of cells overexpressing TRPC5 or TRPC6 with 5 μM of test compound following Carbachol
stimulation.

| | Inhibition of TRPC5 | Inhibition of TRPC6 |
|---|---|---|
| | (A) | (A) |
| 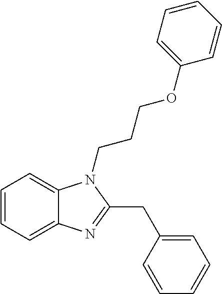 [SMILES: c1ccc(Cc2nc3ccccc3n2CCCOc2ccccc2)cc1] | 85.90 | 92.48 |
| 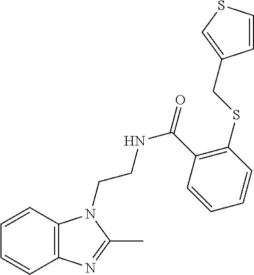 [SMILES: Cc1nc2ccccc2n1CCNC(=O)c1ccccc1SCc1ccsc1] | 77.72 | 80.77 |
| 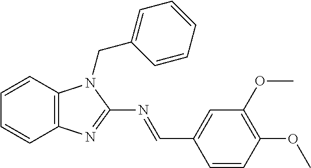 [SMILES: COc1ccc(/C=N/c2nc3ccccc3n2Cc2ccccc2)cc1OC] | 74.31 | 90.52 |
| 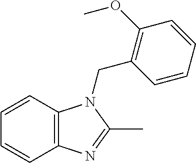 [SMILES: COc1ccccc1Cn1c(C)nc2ccccc21] | 73.39 | 89.89 |
| 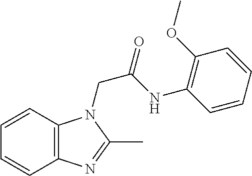 [SMILES: COc1ccccc1NC(=O)Cn1c(C)nc2ccccc21] | 72.70 | 84.09 |
| 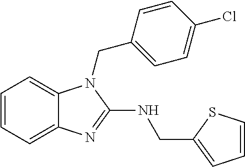 [SMILES: Clc1ccc(Cn2c(NCc3cccs3)nc3ccccc32)cc1] | 72.03 | 37.54 |
| 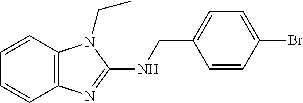 [SMILES: CCn1c(NCc2ccc(Br)cc2)nc2ccccc21] | 71.57 | 77.3 |
| 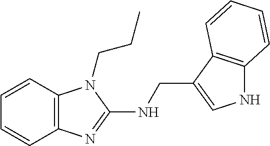 [SMILES: CCCn1c(NCc2c[nH]c3ccccc23)nc2ccccc21] | 70.64 | 54.72 |
| 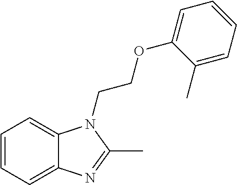 [SMILES: Cc1ccccc1OCCn1c(C)nc2ccccc21] | 70.02 | 76.25 |
| 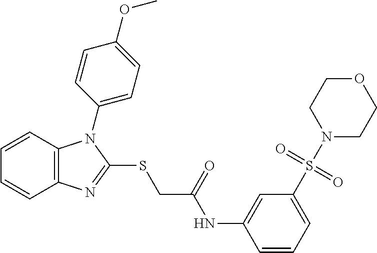 [SMILES: COc1ccc(-n2c(SCC(=O)Nc3cccc(S(=O)(=O)N4CCOCC4)c3)nc3ccccc32)cc1] | 69.61 | 6.92 |
| 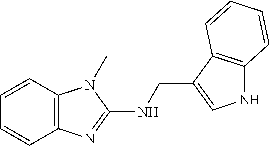 [SMILES: Cn1c(NCc2c[nH]c3ccccc23)nc2ccccc21] | 66.85 | 27.73 |
| 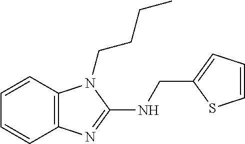 [SMILES: CCCCn1c(NCc2cccs2)nc2ccccc21] | 66.12 | 81.27 |
| 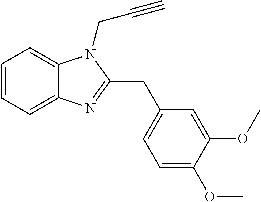 [SMILES: C#CCn1c(Cc2ccc(OC)c(OC)c2)nc2ccccc21] | 65.97 | 91.51 |
| 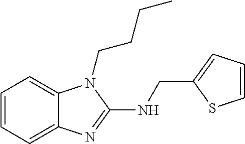 [SMILES: CCCCn1c(NCc2cccs2)nc2ccccc21] | 65.84 | 72.31 |
| 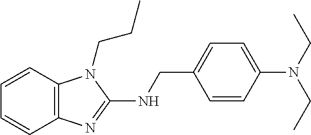 [SMILES: CCCn1c(NCc2ccc(N(CC)CC)cc2)nc2ccccc21] | 64.93 | 74.66 |
| 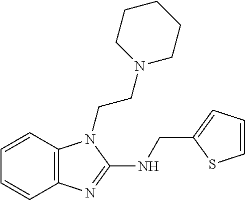 [SMILES: c1csc(CNc2nc3ccccc3n2CCN2CCCCC2)c1] | 64.44 | 16.54 |
| 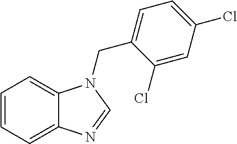 [SMILES: Clc1ccc(Cn2cnc3ccccc32)c(Cl)c1] | 63.51 | 85.46 |
| 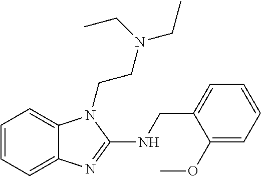 [SMILES: CCN(CC)CCn1c(NCc2ccccc2OC)nc2ccccc21] | 63.38 | 21.95 |
| 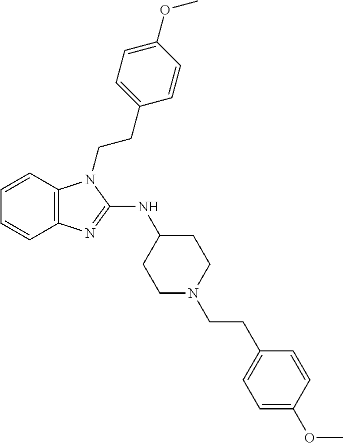 [SMILES: COc1ccc(CCN2CCC(Nc3nc4ccccc4n3CCc3ccc(OC)cc3)CC2)cc1] | 63.34 | 68.88 |
| 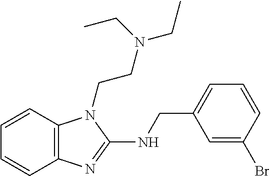 [SMILES: CCN(CC)CCn1c(NCc2cccc(Br)c2)nc2ccccc21] | 62.92 | 69.75 |
| 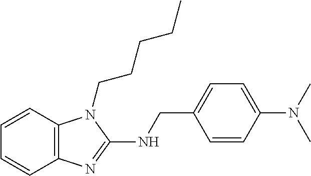 [SMILES: CCCCCn1c(NCc2ccc(N(C)C)cc2)nc2ccccc21] | 62.70 | 81.87 |
| 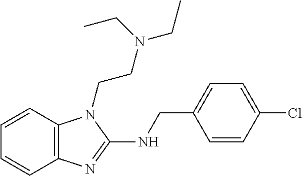 [SMILES: CCN(CC)CCn1c(NCc2ccc(Cl)cc2)nc2ccccc21] | 61.95 | 57.13 |
| 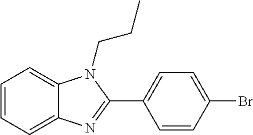 [SMILES: CCCn1c(-c2ccc(Br)cc2)nc2ccccc21] | 61.40 | 82.79 |
| 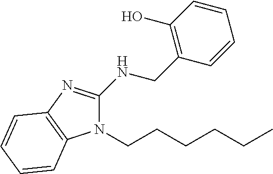 [SMILES: CCCCCCn1c(NCc2ccccc2O)nc2ccccc21] | 61.30 | 21.87 |
| 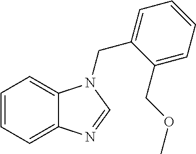 [SMILES: COCc1ccccc1Cn1cnc2ccccc21] | 61.28 | 83.54 |
| 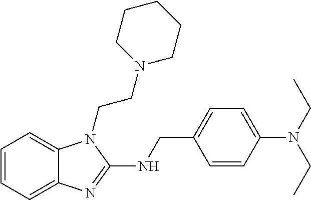 [SMILES: CCN(CC)c1ccc(CNc2nc3ccccc3n2CCN2CCCCC2)cc1] | 61.18 | 65.65 |
| 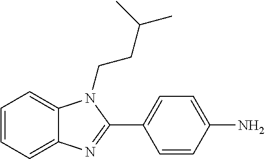 [SMILES: CC(C)CCn1c(-c2ccc(N)cc2)nc2ccccc21] | 61.15 | 27.17 |
| 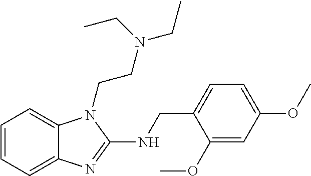 [SMILES: CCN(CC)CCn1c(NCc2ccc(OC)cc2OC)nc2ccccc21] | 60.89 | 43.54 |
| 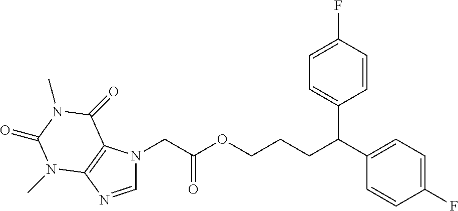 [SMILES: Cn1c(=O)c2c(ncn2CC(=O)OCCCC(c2ccc(F)cc2)c2ccc(F)cc2)n(C)c1=O] | 59.95 | 60.71 |
| 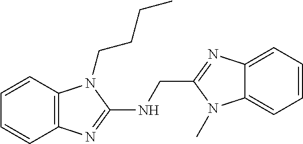 [SMILES: CCCCn1c(NCc2nc3ccccc3n2C)nc2ccccc21] | 59.55 | 30.35 |
| 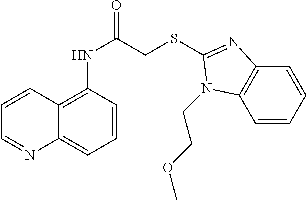 [SMILES: COCCn1c(SCC(=O)Nc2cccc3ncccc23)nc2ccccc21] | 59.09 | 83.61 |
| 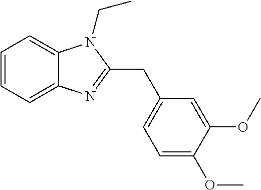 [SMILES: CCn1c(Cc2ccc(OC)c(OC)c2)nc2ccccc21] | 58.97 | 93.97 |
| 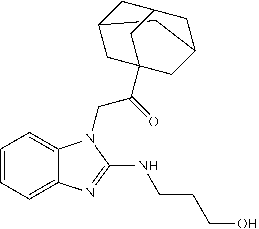 [SMILES: O=C(Cn1c(NCCCO)nc2ccccc21)C12CC3CC(CC(C3)C1)C2] | 58.20 | 28.45 |
| 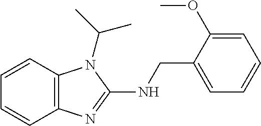 [SMILES: COc1ccccc1CNc1nc2ccccc2n1C(C)C] | 57.91 | 53.90 |
| 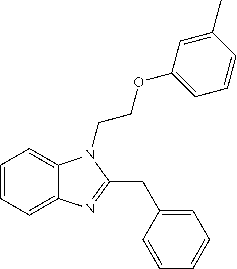 [SMILES: Cc1cccc(OCCn2c(Cc3ccccc3)nc3ccccc32)c1] | 56.35 | 52.88 |
| 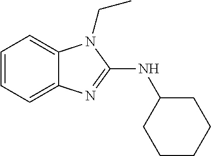 [SMILES: CCn1c(NC2CCCCC2)nc2ccccc21] | 55.45 | 9.58 |
| 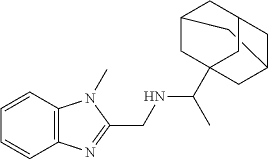 [SMILES: CC(NCc1nc2ccccc2n1C)C12CC3CC(CC(C3)C1)C2] | 55.25 | 69.70 |
| 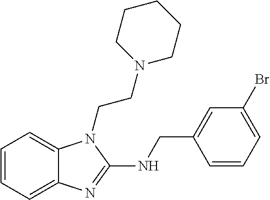 [SMILES: Brc1cccc(CNc2nc3ccccc3n2CCN2CCCCC2)c1] | 55.01 | 50.96 |
| 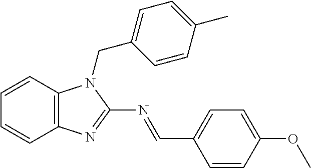 [SMILES: COc1ccc(/C=N/c2nc3ccccc3n2Cc2ccc(C)cc2)cc1] | 53.44 | 41.46 |
| 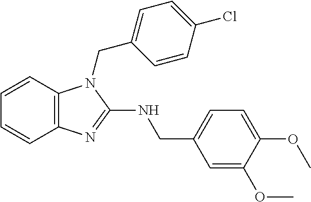 [SMILES: COc1ccc(CNc2nc3ccccc3n2Cc2ccc(Cl)cc2)cc1OC] | 53.42 | 31.26 |
| 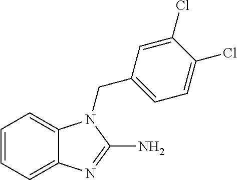 [SMILES: Nc1nc2ccccc2n1Cc1ccc(Cl)c(Cl)c1] | 52.36 | 84.33 |
| 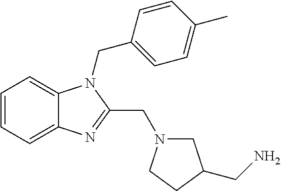 [SMILES: Cc1ccc(Cn2c(CN3CCC(CN)C3)nc3ccccc32)cc1] | 51.81 | 51.04 |
| 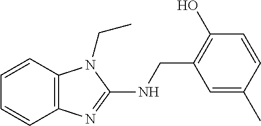 [SMILES: CCn1c(NCc2cc(C)ccc2O)nc2ccccc21] | 51.77 | 3.16 |
| 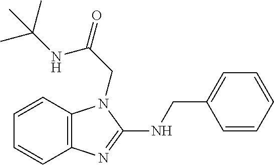 [SMILES: CC(C)(C)NC(=O)Cn1c(NCc2ccccc2)nc2ccccc21] | 51.04 | 3.97 |
| 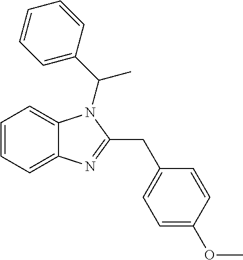 [SMILES: COc1ccc(Cc2nc3ccccc3n2C(C)c2ccccc2)cc1] | 51.03 | 44.98 |
| 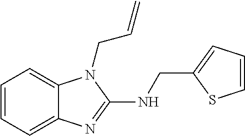 [SMILES: C=CCn1c(NCc2cccs2)nc2ccccc21] | 50.70 | 14.59 |
| 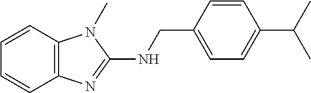 [SMILES: CC(C)c1ccc(CNc2nc3ccccc3n2C)cc1] | 50.02 | 80.58 |
| 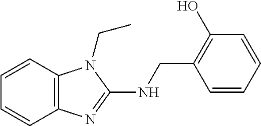 [SMILES: CCn1c(NCc2ccccc2O)nc2ccccc21] | 49.99 | −8.69 |
| 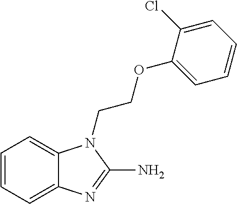 [SMILES: Nc1nc2ccccc2n1CCOc1ccccc1Cl] | 49.87 | 53.73 |
| 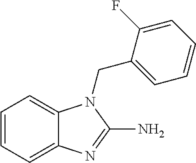 [SMILES: Nc1nc2ccccc2n1Cc1ccccc1F] | 48.39 | 34.78 |
| 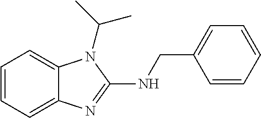 [SMILES: CC(C)n1c(NCc2ccccc2)nc2ccccc21] | 48.36 | 22.42 |
| 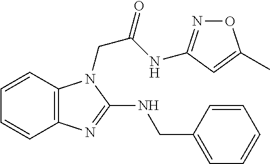 [SMILES: Cc1cc(NC(=O)Cn2c(NCc3ccccc3)nc3ccccc32)no1] | 48.01 | 22.81 |
| 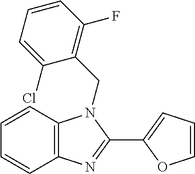 [SMILES: Fc1cccc(Cl)c1Cn1c(-c2ccco2)nc2ccccc21] | 47.92 | 61.73 |
| 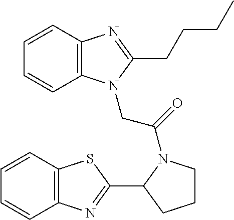 [SMILES: CCCCc1nc2ccccc2n1CC(=O)N1CCCC1c1nc2ccccc2s1] | 46.27 | −0.71 |
| 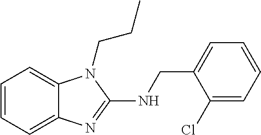 [SMILES: CCCn1c(NCc2ccccc2Cl)nc2ccccc21] | 45.99 | 29.82 |
| 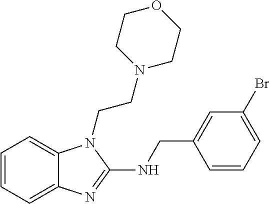 [SMILES: Brc1cccc(CNc2nc3ccccc3n2CCN2CCOCC2)c1] | 45.80 | 18.52 |
| 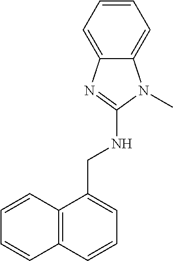 [SMILES: Cn1c(NCc2cccc3ccccc23)nc2ccccc21] | 45.46 | 27.46 |
| 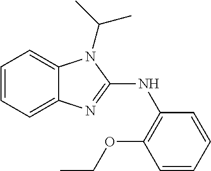 [SMILES: CCOc1ccccc1Nc1nc2ccccc2n1C(C)C] | 45.15 | 40.7 |
| 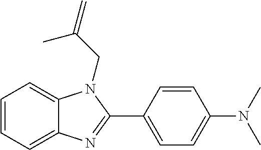 [SMILES: C=C(C)Cn1c(-c2ccc(N(C)C)cc2)nc2ccccc21] | 45.02 | 44.96 |
| 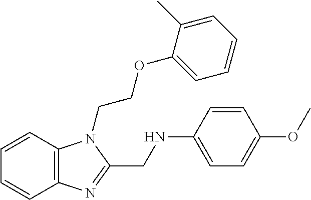 [SMILES: COc1ccc(NCc2nc3ccccc3n2CCOc2ccccc2C)cc1] | 44.89 | 58.46 |
| 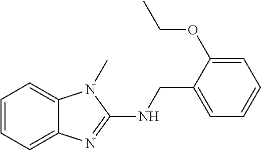 [SMILES: CCOc1ccccc1CNc1nc2ccccc2n1C] | 43.92 | 19.3 |
| 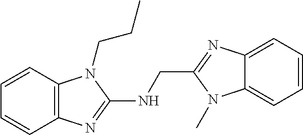 [SMILES: CCCn1c(NCc2nc3ccccc3n2C)nc2ccccc21] | 42.82 | −6.21 |
| 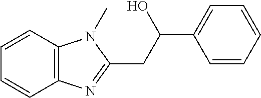 [SMILES: Cn1c(CC(O)c2ccccc2)nc2ccccc21] | 42.29 | −9.99 |
| 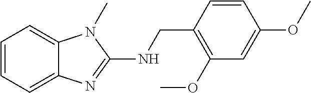 [SMILES: COc1ccc(CNc2nc3ccccc3n2C)c(OC)c1] | 41.31 | 19.39 |
| 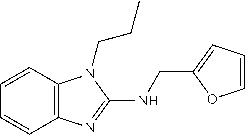 [SMILES: CCCn1c(NCc2ccco2)nc2ccccc21] | 40.68 | 14.88 |
| 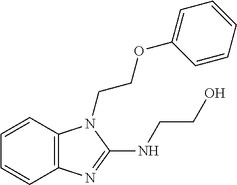 [SMILES: OCCNc1nc2ccccc2n1CCOc1ccccc1] | 40.68 | 8.36 |
| 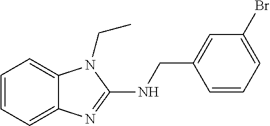 [SMILES: CCn1c(NCc2cccc(Br)c2)nc2ccccc21] | 39.93 | 54.15 |
| 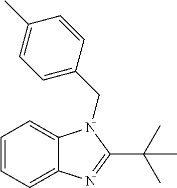 [SMILES: Cc1ccc(Cn2c(C(C)(C)C)nc3ccccc32)cc1] | 39.72 | 27.72 |
| 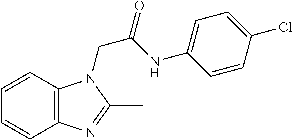 [SMILES: Cc1nc2ccccc2n1CC(=O)Nc1ccc(Cl)cc1] | 39.70 | 70.00 |
| 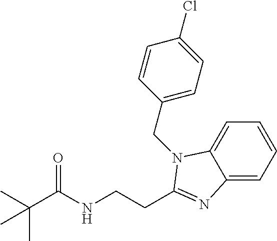 [SMILES: CC(C)(C)C(=O)NCCc1nc2ccccc2n1Cc1ccc(Cl)cc1] | 38.94 | −7.07 |
| 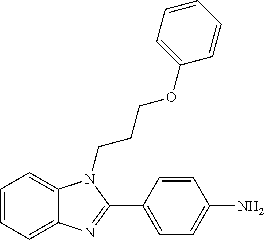 [SMILES: Nc1ccc(-c2nc3ccccc3n2CCCOc2ccccc2)cc1] | 38.59 | 27.31 |
| 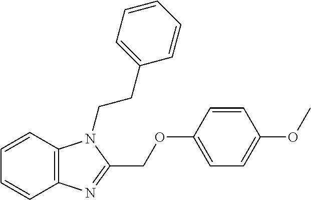 [SMILES: COc1ccc(OCc2nc3ccccc3n2CCc2ccccc2)cc1] | 38.29 | 51.96 |
| 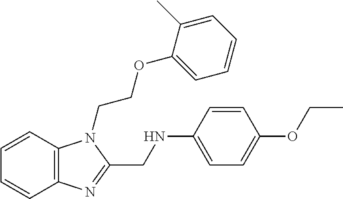 [SMILES: CCOc1ccc(NCc2nc3ccccc3n2CCOc2ccccc2C)cc1] | 37.45 | 66.09 |
| 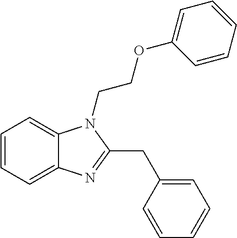 [SMILES: c1ccc(Cc2nc3ccccc3n2CCOc2ccccc2)cc1] | 36.66 | 17.71 |
| 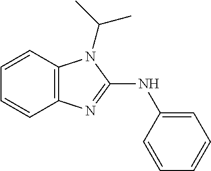 [SMILES: CC(C)n1c(Nc2ccccc2)nc2ccccc21] | 35.66 | 14.47 |
| 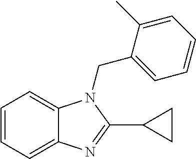 [SMILES: Cc1ccccc1Cn1c(C2CC2)nc2ccccc21] | 35.53 | 45.28 |
| 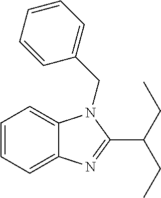 [SMILES: CCC(CC)c1nc2ccccc2n1Cc1ccccc1] | 34.87 | 35.28 |
| 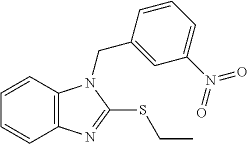 [SMILES: CCSc1nc2ccccc2n1Cc1cccc([N+](=O)[O-])c1] | 32.17 | 50.03 |
| 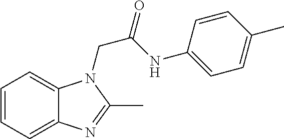 [SMILES: Cc1ccc(NC(=O)Cn2c(C)nc3ccccc32)cc1] | 32.12 | 81.83 |
| 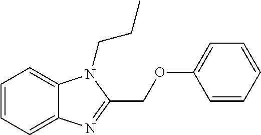 [SMILES: CCCn1c(COc2ccccc2)nc2ccccc21] | 30.18 | 22.8 |
| 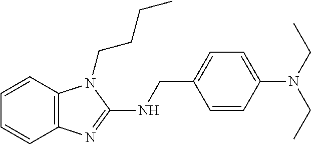 [SMILES: CCCCn1c(NCc2ccc(N(CC)CC)cc2)nc2ccccc21] | 28.74 | 30.29 |
| 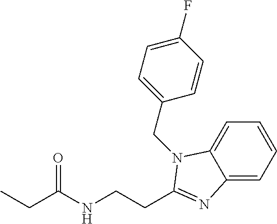 [SMILES: CCC(=O)NCCc1nc2ccccc2n1Cc1ccc(F)cc1] | 27.00 | 2.78 |

What is claimed is:

1. A compound of Formula (I) or (II) or a pharmaceutically acceptable salt thereof; 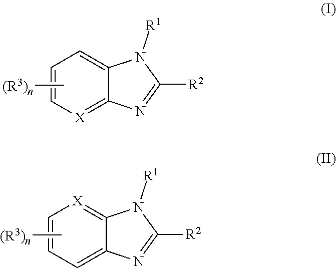
wherein
X is CH, C(R³), or N;
R¹ is selected from the group consisting of H; alkyl; cycloalkyl; heterocycloalkyl; aryl; heteroaryl; alkenyl; alkylene-aryl; alkylene-heteroaryl; —CH₂(O)N(R)-heteroaryl; —CH₂(O)N(R)-alkyl; alkylene-N(alkyl)₂; heterocycloalkyl; alkylene-O-alkyl; alkylene-O-aryl; alkylene-N(R)—C(O)-aryl; alkylene-N(R)—C(O)-alkyl; alkylene-C(O)—N(R)-alkyl; alkylene-C(O)—N(R)-aryl; alkylene-C(O)-cycloalkyl; and alkylene-C(O)—N(R)-heteroaryl;
R² is selected from the group consisting of H; NH₂; alkyl; cycloalkyl; aryl; heteroaryl; alkylene-aryl; alkylene-N(alkyl)₂; alkylene-heterocycloalkyl; alkylene-cycloalkyl; —N(R)-alkyl; —N(R)-aryl; —N(R)-alkylene-aryl; —N(R)-cycloalkyl; —N(R)-heterocycloalkyl; —O-aryl; alkylene-O-aryl; heterocycloalkyl; —N═C(R)-aryl; —N(R)-alkylene-heteroaryl; —N(R)-alkylene-OH; —S— alkylene-C(O)N(R)-aryl; —S-alkylene-C(O)N(R)-heteroaryl; alkylene-C(O)-heterocycloalkyl; alkylene-N(R)-alkyl; alkylene-N(R)-aryl; and —S-alkyl;
R³ is independently selected from alkyl, halogen, CN, OMe, OH, NO₂, NH₂, N(Me)₂, CF₃, OCF₃, CHF₂, OCHF₂, and —O-alkylene-OH;
R is H, or Me; and
n is 0, 1, 2, 3, or 4;
provided the compound is not

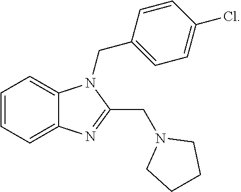 [SMILES: Clc1ccc(Cn2c(CN3CCCC3)nc3ccccc32)cc1]

2. The compound of claim 1, wherein R¹ is selected from the group consisting of H, methyl, ethyl, n-propyl, i-propyl, n-butyl, i-butyl, and t-butyl.

3. The compound of claim 1, wherein R¹ is selected from the group consisting of alkylene-aryl, and alkylene-heteroaryl.

4. The compound of claim 3, wherein alkylene-aryl is methylene-phenyl.

5. The compound of claim 4, wherein the phenyl is substituted with alkyl, halogen, OMe, OH, NO₂, NH₂, N(Me)₂, CF₃, OCF₃, CHF₂, OCHF₂, or —O-alkylene-OH.

6. The compound of claim 3, wherein alkylene-heteroaryl is methylene-pyridine.

7. The compound of claim 6, wherein the pyridine is substituted with alkyl, halogen, OMe, OH, NO₂, NH₂, N(Me)₂, CF₃, OCF₃, CHF₂, OCHF₂, or —O-alkylene-OH.

8. The compound of claim 1, wherein R¹ is selected from —CH₂(O)N(R)-heteroaryl; —CH₂(O)N(R)-alkyl.

9. The compound of claim 8, wherein R¹ is —CH₂(O)N(H)-heteroaryl.

10. The compound of claim 9, wherein heteroaryl is substituted oxazole.

11. The compound of claim 8, wherein R¹ is —CH₂(O)N(H)-alkyl.

12. The compound of claim 11, wherein alkyl is t-butyl.

13. The compound of claim 1, wherein R¹ is alkylene-N(Me)₂ or alkylene-N(Et)₂.

14. The compound of claim 13, wherein alkylene is ethylene.

15. The compound of claim 1, wherein R is heterocycloalkyl.

16. The compound of claim 15, wherein heterocycloalkyl, is methyl-piperidinyl.

17. The compound of claim 1, wherein R¹ is alkenyl.

18. The compound of claim 1, wherein R¹ is substituted aryl.

19. The compound of claim 1, wherein R is selected from alkylene-O-alkyl, and alkylene-O-aryl.

20. The compound of claim 1, wherein R¹ is alkylene-C(O)-cycloalkyl.

21. The compound of claim 1, wherein R¹ is selected from alkylene-N(H)—C(O)-aryl, and alkylene-N(H)—C(O)-alkyl.

22. The compound of claim 1, wherein R¹ is selected from alkylene-C(O)—N(H)-alkyl, alkylene-C(O)—N(H)-aryl, and alkylene-C(O)—N(H)-heteroaryl

23. The compound of any one of claims 1-22, wherein R² is NH₂.

24. The compound of any one of claims 1-22, wherein R² is alkylene-aryl.

25. The compound of claim 24, wherein alkylene-aryl is methylene-phenyl.

26. The compound of claim 25, wherein the phenyl is substituted with alkyl, halogen, OMe, OH, NO₂, NH₂, N(Me)₂, CF₃, OCF₃, CHF₂, OCHF₂, or —O-alkylene-OH.

27. The compound of any one of claims 1-22, wherein R² is alkylene-N(Me)₂.

28. The compound of claim 27, wherein alkylene is ethylene.

29. The compound of any one of claims 1-22, wherein R² is selected —N(H)-alkyl; —N(H)-aryl; —N(H)-cycloalkyl; —N(H)-heterocycloalkyl.

30. The compound of claim 29, wherein alkyl is methyl, ethyl, n-propyl, i-propyl, n-butyl, i-butyl, or t-butyl.

31. The compound of claim 29, wherein alkyl is methyl, or butyl.

32. The compound of claim 29, wherein aryl is phenyl.

33. The compound of claim 29, wherein cycloalkyl is cyclohexyl.

34. The compound of claim 23, wherein heterocycloalkyl is tetrahydropyranyl.

35. The compound of any one of claims 1-22, wherein R² is —N(H)-alkylene-aryl.

36. The compound of claim 29, wherein —N(H)-alkylene-aryl, is —N(H)-methylene-phenyl.

37. The compound of claim 30, wherein the phenyl is substituted with alkyl, halogen, OMe, OH, NO₂, NH₂, N(Me)₂, CF₃, OCF₃, CHF₂, OCHF₂, or —O-alkylene-OH.

38. The compound of any one of claims 1-15, wherein R² is —O-phenyl.

39. The compound of claim 32, wherein the phenyl is substituted with halogen.

40. The compound of any one of claims 1-15, wherein R² is heterocycloalkyl.

41. The compound of claim 40, wherein heterocycloalkyl is piperidinyl, pyrrolidinyl, or pyrrolidinonyl.

42. The compound of claim 41, wherein piperidinyl is substituted piperidinyl.

43. The compound of claim 42, wherein pyrrolidinyl is substituted pyrrolidinyl.

44. The compound of claim 42, wherein pyrrolidinyl is substituted pyrrolidinonyl.

45. The compound of any one of claims 42-44, wherein the substitution is an alkylene-heteroaryl.

46. The compound of claim 45, wherein the heteroaryl is substituted.

47. The compound of any one of claims 43-44, the substitution is an alkylene-aryl.

48. The compound of claim 47, wherein the aryl is substituted.

49. The compound of any one of claims 1-15, wherein R² is H.

50. The compound of any one of claims 1-15, wherein R² is alkyl or cycloalkyl.

51. The compound of claim 50, wherein alkyl is selected from methyl, ethyl, n-propyl, i-propyl, n-butyl, i-butyl, and t-butyl.

52. The compound of claim 50, wherein cycloalkyl is cyclopropyl.

53. The compound of any one of claims 1-15, wherein R² is aryl.

54. The compound of claim 53, wherein aryl is substituted phenyl.

55. The compound of any one of claims 1-15, wherein R² is —O-aryl and alkylene-O-aryl.

56. The compound of claim 55, wherein the aryl is substituted.

57. The compound of any one of claims 1-15, wherein R² is N═C(H)-aryl.

58. The compound of claim 57, wherein the aryl is substituted.

59. The compound of any one of claims 1-15, wherein R² is —N(H)-alkylene-heteroaryl, or —N(H)-alkylene-OH.

60. The compound of any one of claims 1-15, wherein R² is —S-alkylene-C(O)N(H)-aryl, or —S— alkylene-C(O)N(H)-heteroaryl;

61. The compound of any one of claims 1-15, wherein R² is alkylene-C(O)-heterocycloalkyl;

62. The compound of claim 61, wherein heterocycloalkyl is substituted with heteroaryl.

63. The compound of any one of claims 1-15, wherein R² is alkylene-N(H)-alkyl, or alkylene-N(H)-aryl.

64. The compound of claim 63, wherein the alkyl is substituted.

65. The compound of claim 64, wherein the alkyl is substituted with cycloalkyl.

66. The compound of claim 63, wherein the aryl is substituted.

67. The compound of any one of claims 1-15, wherein R² is —S-alkyl.

68. The compound of any one of claims 1-15, wherein R² is methylene-cycloalkyl.

69. The compound of any one of claims 1-15, wherein R² is methylene-heterocycloalkyl.

70. The compound of any one of claim 1-69, wherein R³ is methyl.

71. The compound of any one of claim 1-69, wherein R³ is F.

72. The compound of any one of claims 1-69, wherein n is 0.

73. The compound of any one of claims 1-71, wherein n is 1.

74. The compound of any one of claims 1-73, wherein X is CH.

75. The compound of any one of claims 1-73, wherein X is C(R³).

76. The compound of any one of claims 1-73, wherein X is N.

77. The compound of claim 1, wherein the compound is selected from the group consisting of:

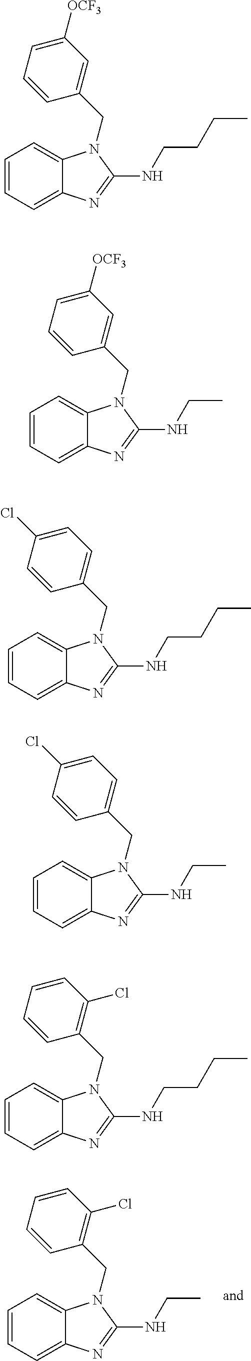

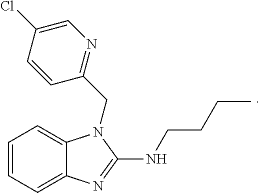 [SMILES: CCCCNc1nc2ccccc2n1Cc1ccc(Cl)cn1]

78. The compound of claim 1, wherein the compound is selected from the group consisting of:

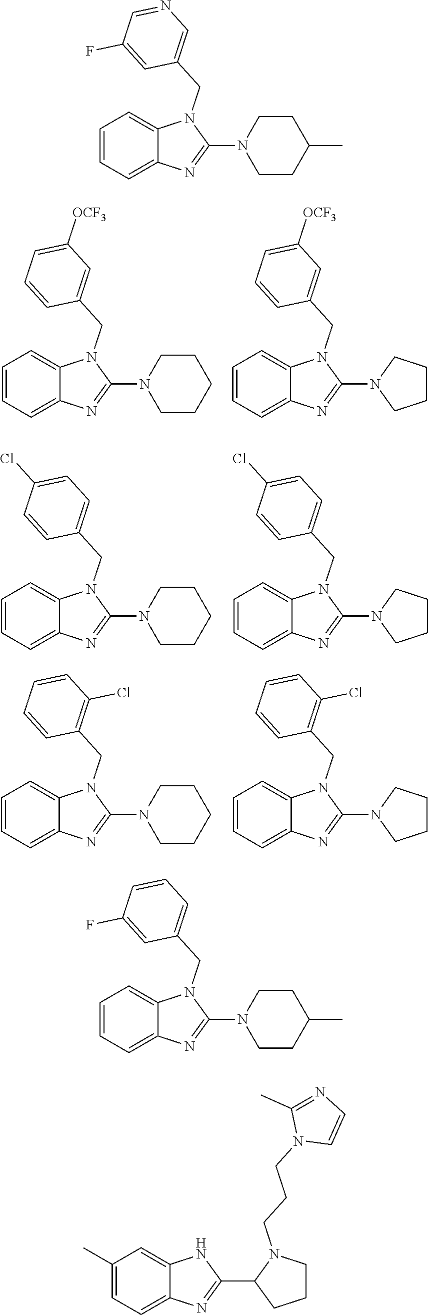

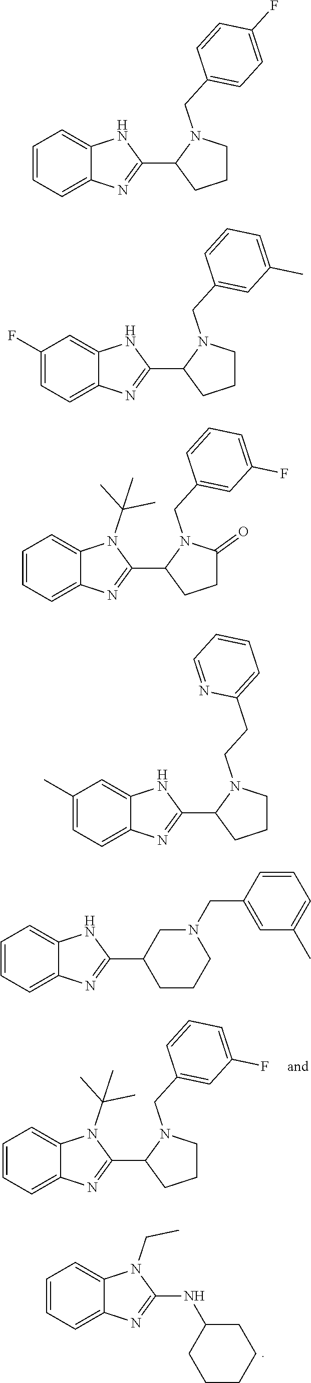

79. The compound of claim 1, wherein the compound is selected from the group consisting of:

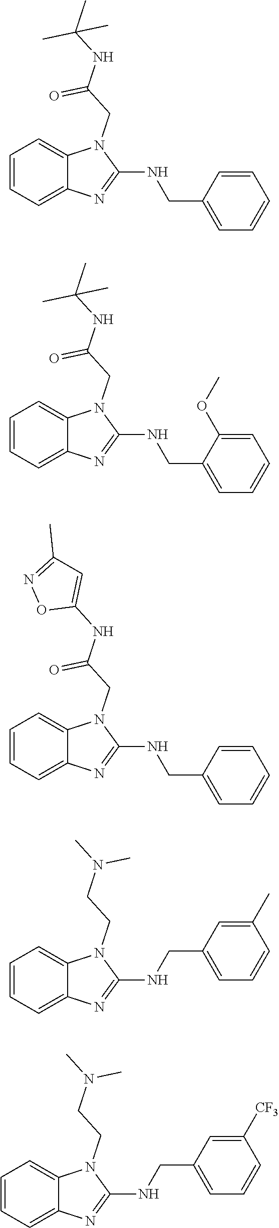 [SMILES: CC(C)(C)NC(=O)Cn1c(NCc2ccccc2)nc2ccccc21 ; COc1ccccc1CNc1nc2ccccc2n1CC(=O)NC(C)(C)C ; Cc1cc(NC(=O)Cn2c(NCc3ccccc3)nc3ccccc32)on1 ; Cc1cccc(CNc2nc3ccccc3n2CCN(C)C)c1 ; CN(C)CCn1c(NCc2cccc(C(F)(F)F)c2)nc2ccccc21]

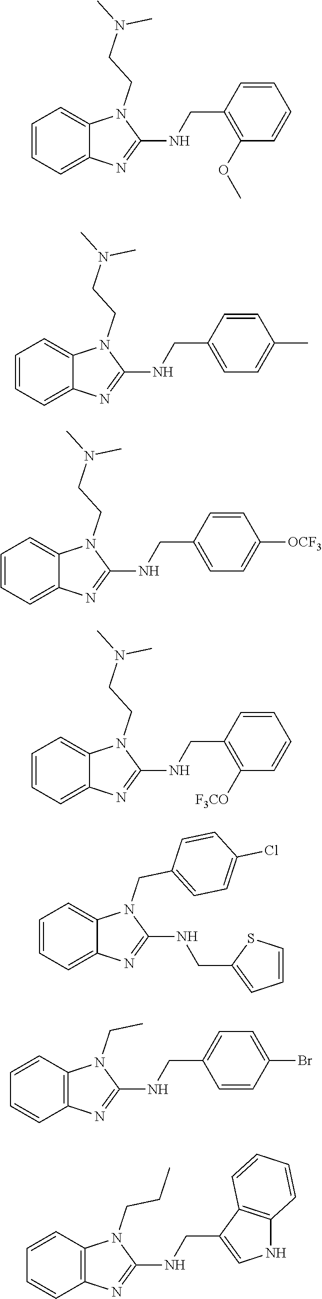

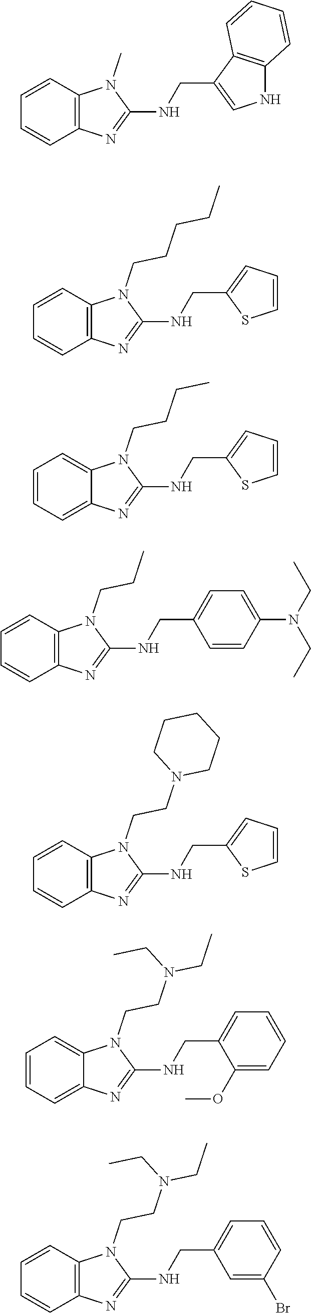

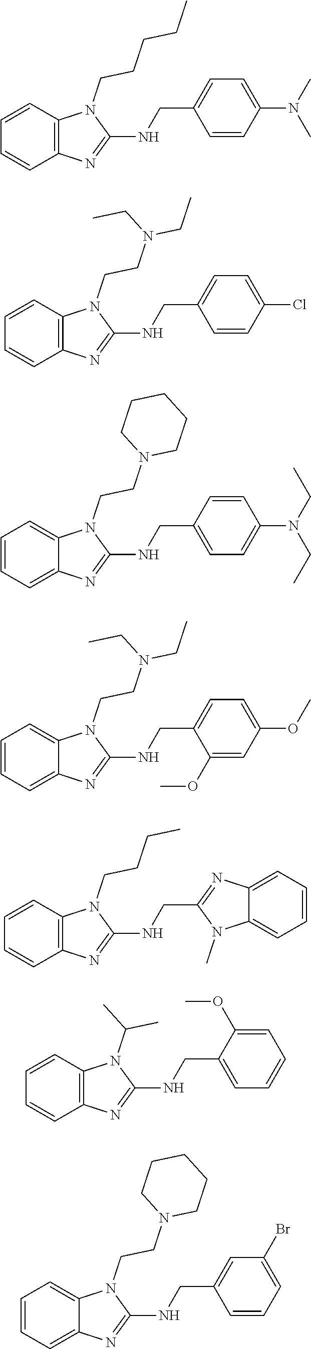

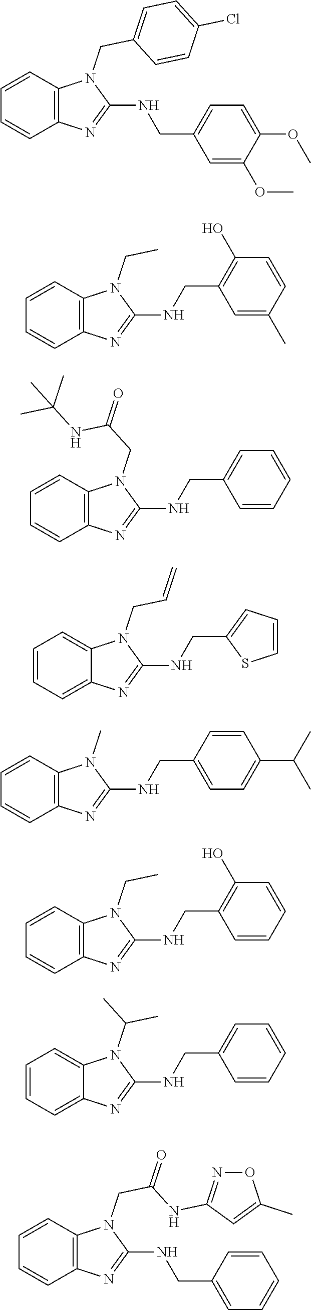

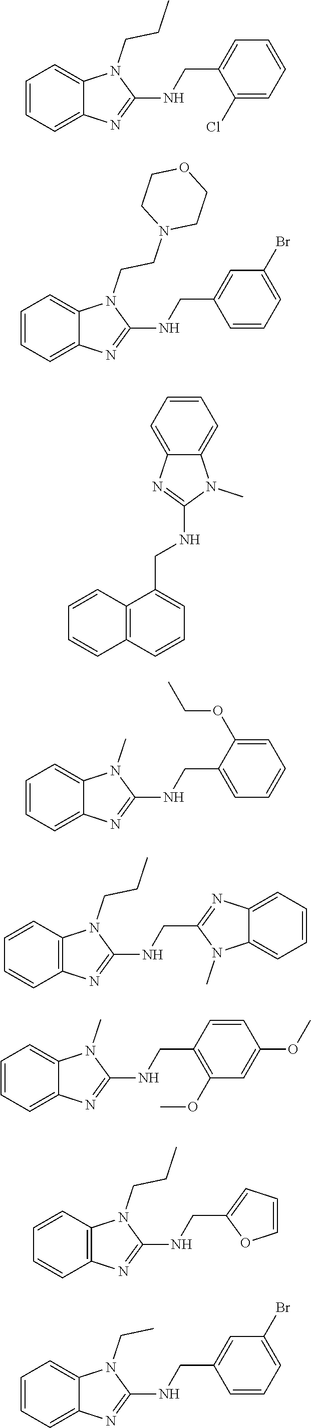

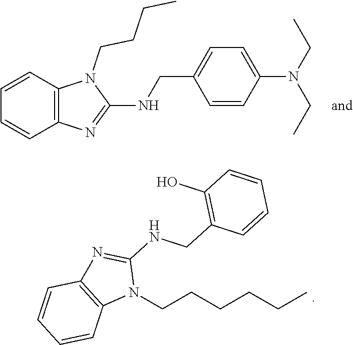 [SMILES: CCCCn1c(NCc2ccc(N(CC)CC)cc2)nc2ccccc21 ; CCCCCCn1c(NCc2ccccc2O)nc2ccccc21]

80. The compound of claim 1, wherein the compound is selected from the group consisting of:

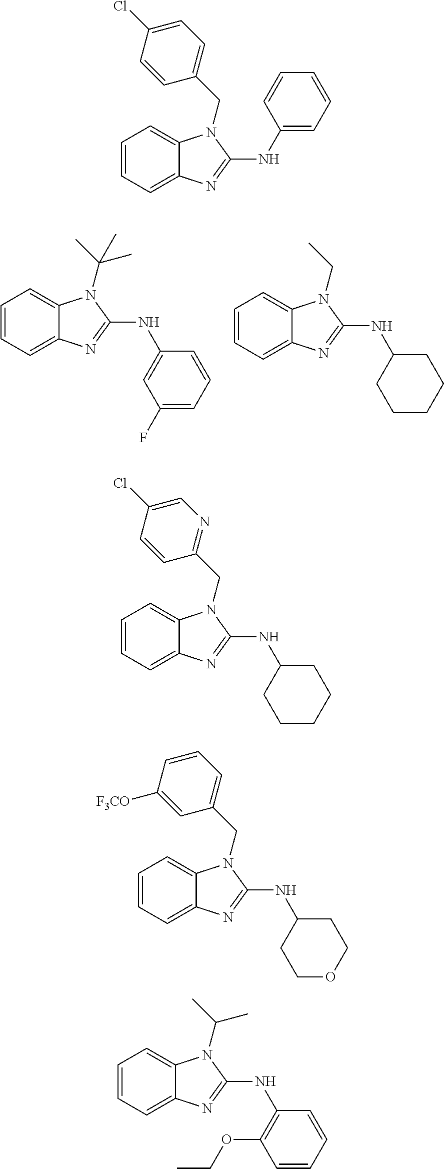

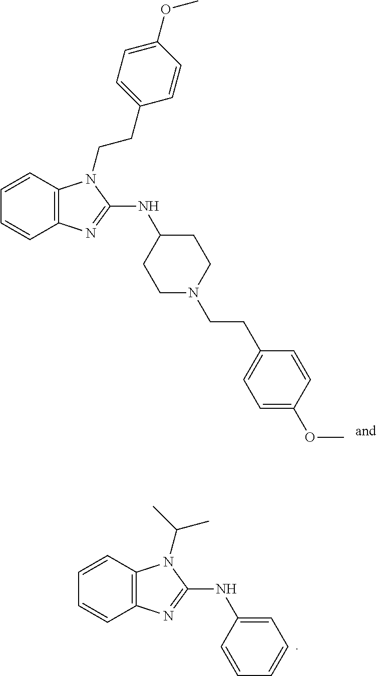 [SMILES: COc1ccc(CCN2CCC(Nc3nc4ccccc4n3CCc3ccc(OC)cc3)CC2)cc1 ; CC(C)n1c(Nc2ccccc2)nc2ccccc21]

81. The compound of claim 1, wherein the compound is selected from the group consisting of:

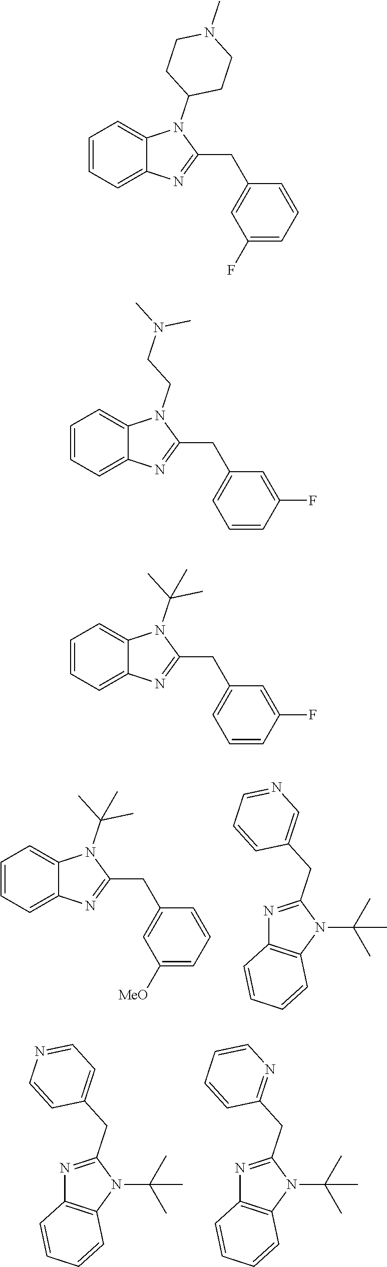

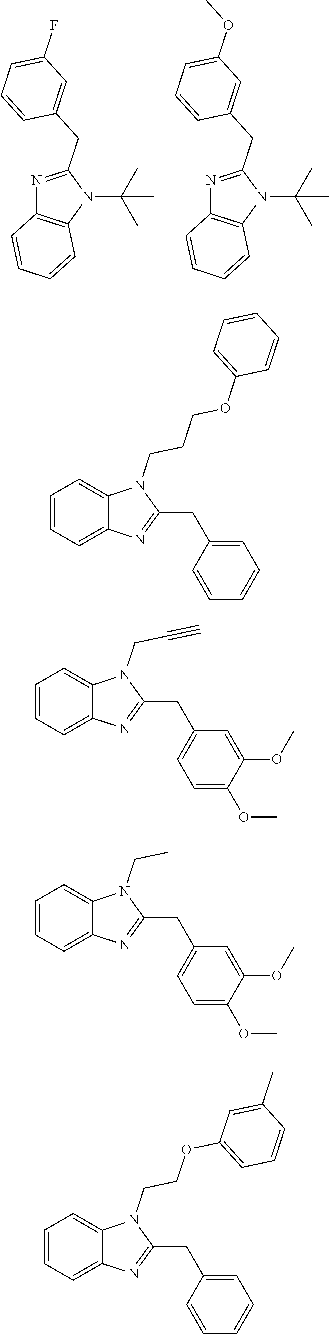

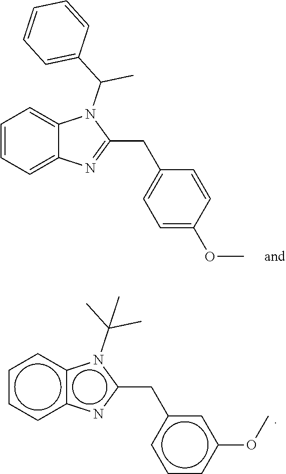

82. The compound of claim 1, wherein the compound is selected from the group consisting of:

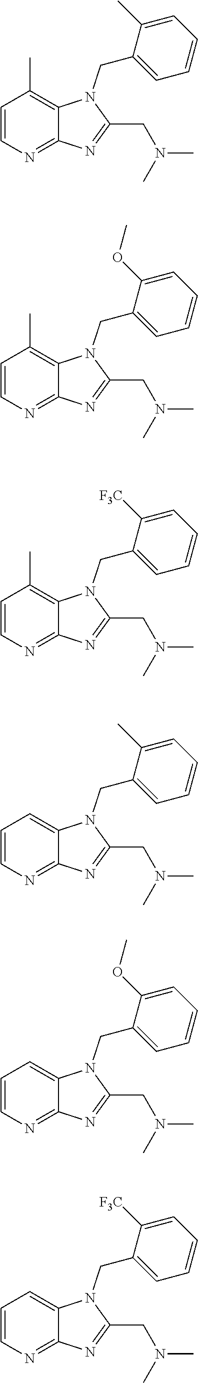

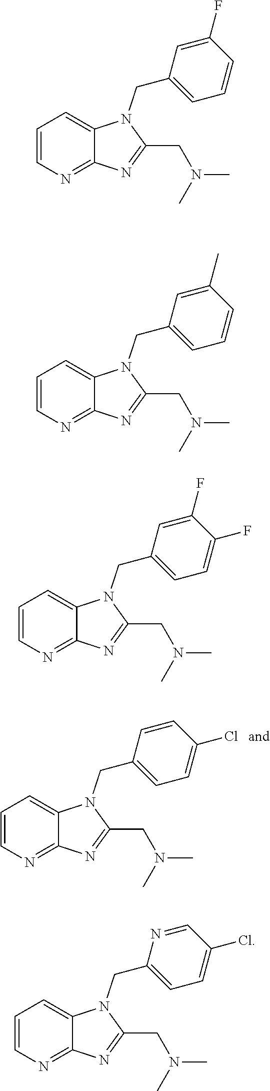

83. The compound of claim 1, wherein the compound is selected from the group consisting of:

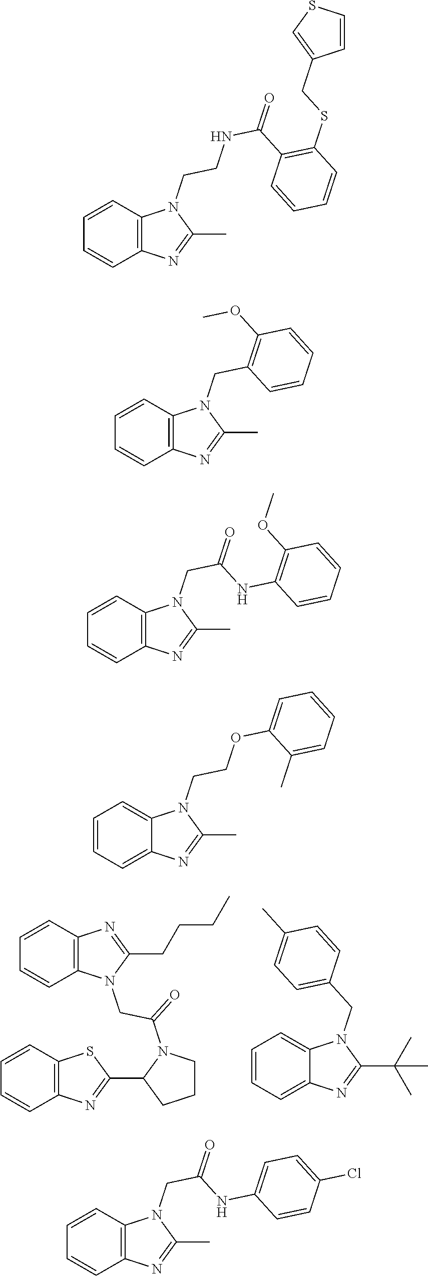

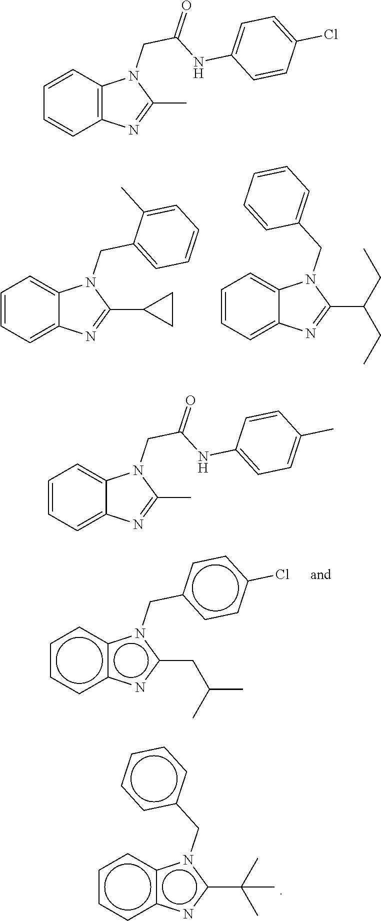

84. The compound of claim 1, wherein the compound is selected from the group consisting of:

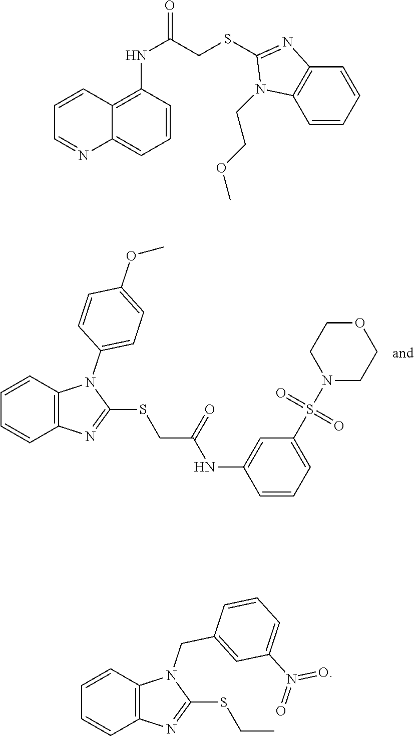 [SMILES: COCCn1c(SCC(=O)Nc2cccc3ncccc23)nc2ccccc21 ; COc1ccc(-n2c(SCC(=O)Nc3cccc(S(=O)(=O)N4CCOCC4)c3)nc3ccccc32)cc1 ; CCSc1nc2ccccc2n1Cc1cccc([N+](=O)[O-])c1]

85. The compound of claim 1, wherein the compound is selected from the group consisting of:

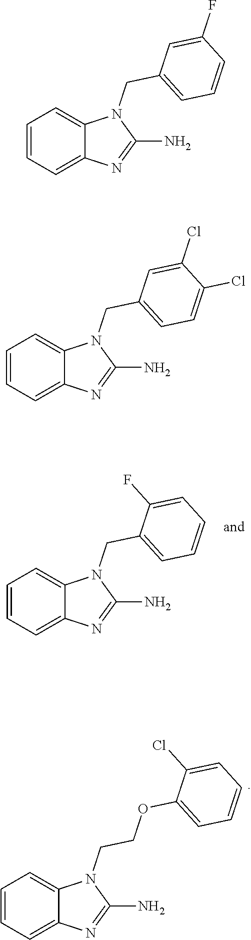 [SMILES: Nc1nc2ccccc2n1Cc1cccc(F)c1 ; Nc1nc2ccccc2n1Cc1ccc(Cl)c(Cl)c1 ; Nc1nc2ccccc2n1Cc1ccccc1F ; Nc1nc2ccccc2n1CCOc1ccccc1Cl]

86. The compound of claim 1, wherein the compound is selected from the group consisting of:

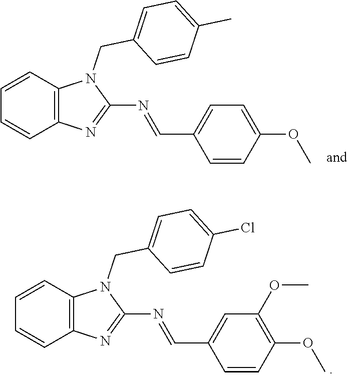 [SMILES: COc1ccc(/C=N/c2nc3ccccc3n2Cc2ccc(C)cc2)cc1 ; COc1ccc(/C=N/c2nc3ccccc3n2Cc2ccc(Cl)cc2)cc1OC]

87. The compound of claim 1, wherein the compound is selected from the group consisting of:

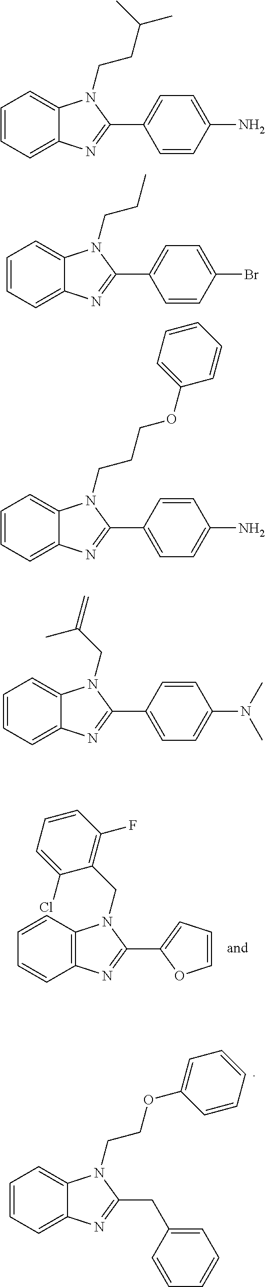

88. The compound of claim 1, wherein the compound is selected from the group consisting of:

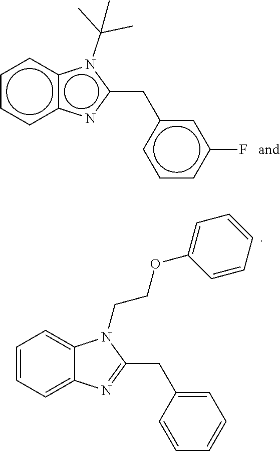

89. The compound of claim 1, wherein the compound is selected from the group consisting of:

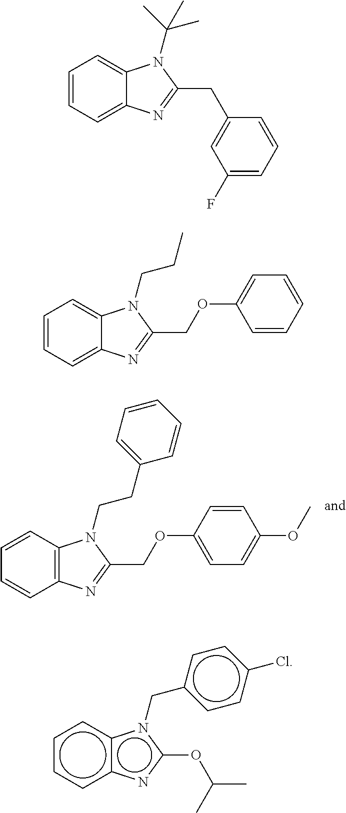

90. The compound of claim 1, wherein the compound is selected from the group consisting of:

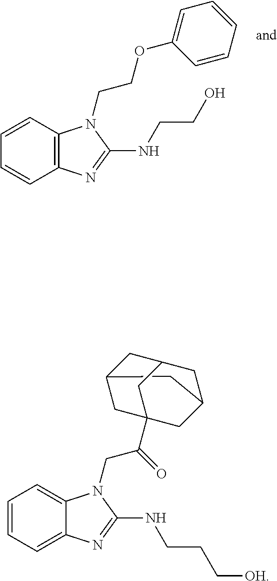 [SMILES: OCCNc1nc2ccccc2n1CCOc1ccccc1 ; O=C(Cn1c(NCCCO)nc2ccccc21)C12CC3CC(CC(C3)C1)C2]

91. The compound of claim 1, wherein the compound is selected from the group consisting of:

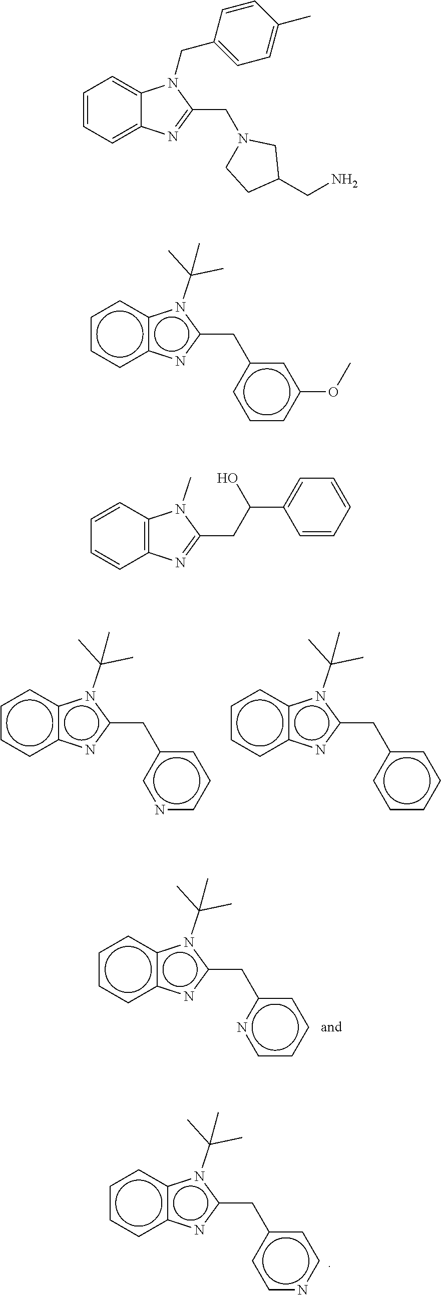

92. The compound of claim 1, wherein the compound is selected from the group consisting of:

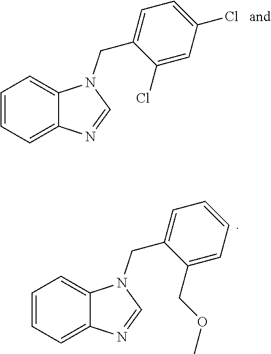

93. The compound of claim 1, wherein the compound is selected from the group consisting of:

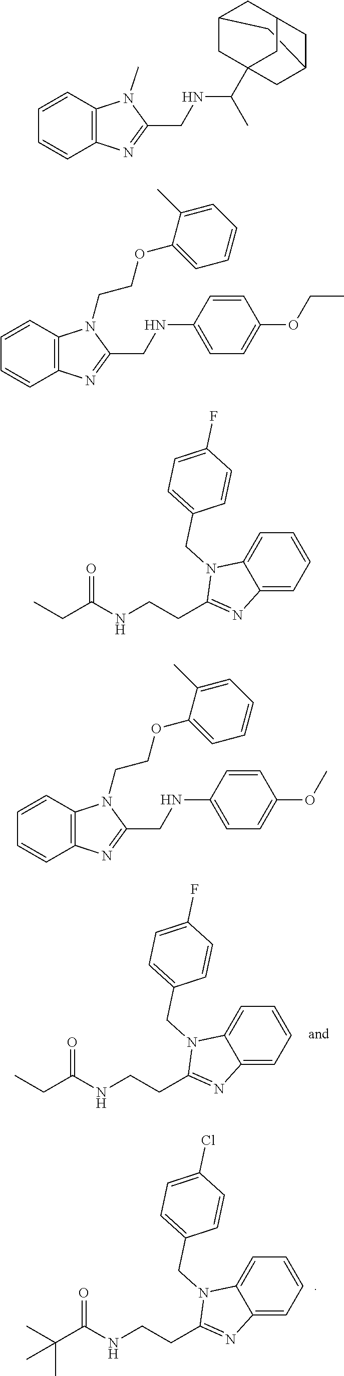

94. A composition, comprising a compound of any one of claims 1-93 or a pharmaceutically acceptable salt thereof; and a pharmaceutically acceptable excipient.

95. A method of treating, or the reducing risk of developing, a kidney disease, anxiety, or depression, or cancer, comprising administering to a subject in need thereof a therapeutically effective amount of a compound of any one of claims 1-93.

96. The method of claim 95, wherein a kidney disease is treated or the risk of developing a kidney disease is reduced.

97. The method of claim 95, wherein a kidney disease is treated.

98. The method of any one of claims 95-97, wherein the kidney disease is selected from the group consisting of Focal Segmental Glomerulosclerosis (FSGS), Diabetic nephropathy, Alport syndrome, hypertensive kidney disease, nephrotic syndrome, steroid-resistant nephrotic syndrome, minimal change disease, membranous nephropathy, idiopathic membranous nephropathy, membranoproliferative glomerulonephritis (MPGN), immune complex-mediated MPGN, complement-mediated MPGN, Lupus nephritis, postinfectious glomerulonephritis, thin basement membrane disease, mesangial proliferative glomerulonephritis, amyloidosis (primary), c1q nephropathy, rapidly progressive GN, anti-GBM disease, C3 glomerulonephritis, hypertensive nephrosclerosis, and IgA nephropathy.

99. The method of any one of claims 95-97, wherein the kidney disease is proteinuria.

100. The method of any one of claims 95-97, wherein the kidney disease is microalbuminuria or macroalbuminuria.

101. The method of any one of claims 95-100, wherein the subject is a mammal

102. The method of claim 101, wherein the mammal is a human.
